SEQUENCE LISTING (29 sequences)

SEQ ID NO: 1 - DNA, 306 bp, Human Immunodeficiency Virus 1
atggagccagtagatcctaaactagagccctggaagcatccaggaagtcagcctaagactgcttgtaacaattgctattgtaaaaagtgttgctttcattgccaagtttgtttcacaaaaaaaggcttaggcatctcctatggcaggaagaagcggagacagcgaagaagagctcctcaagacagtcagactcatcaagcttctctatcaaagcaaccctcctcccagccccgaggggactcgacaggcccgacggaatcgaagaagaaggtggagagagagacagagacagatccgtacgattag

SEQ ID NO: 2 - protein, 101 aa, Human Immunodeficiency Virus 1
MEPVDPRLEPWKHPGSQPKTACTNCYCKKCCFHCQVCFITKALGISYGRKKRRQRRRPPQGSQTHQVSLSKQPTSQSRGDPTGPKESKKKVERETETDPFD

SEQ ID NO: 3 - protein, 101 aa, Human Immunodeficiency Virus 1
MEPVDPNLEPWKHPGSQPKTPCNKCYCKHCSYHCLVCFQKKGLGISYGRKKWRQRRSAPPSSEDHQNPISKQPLPQTRGDPTGSEESKKKVESETKTDPYD

SEQ ID NO: 4 - protein, 101 aa, Human Immunodeficiency Virus 1
MEPVDPKLEPWNHPGSQPKTACNQCFCKACSYHCLVCFQKKGLGTSYGRKKRRQRRSAPPSSEDHQNPISKQPLPQTRGDPTGSEESKKKVENKTKTDQFD

SEQ ID NO: 5 - protein, 101 aa, Human Immunodeficiency Virus 1
MEPVDPNLEPWNHPGSQPKTPCTKCYCKYCSYHCLVCFQTKGLGISYGRKKRRQRRSTPPSSEDHQNPISKQPLPQTRGNSTGSEESKKKVESKTETDPFD

SEQ ID NO: 6 - protein, 100 aa, Human Immunodeficiency Virus 1
MEPVDPNLEPWNHPGSQPKTACNNCFCKHCSYHCLVCFQTKGLGSYGRKKRRQRRSAPPDSEDHQNLISKQPSSQTRGDPTGSEESKKKVESKTKADPFD

SEQ ID NO: 7 - protein, 100 aa, Human Immunodeficiency Virus 1
MEPVDPNLEPWNHPGSQPKTACNNCYCKHCSYHCLVCFQTKGLGSYGRKKRRQRRSAPPSSEDHQNLISKQPLPQTRGDPTGSEESKKKVESKTETDPCD

SEQ ID NO: 8 - protein, 100 aa, Human Immunodeficiency Virus 1
MDPIDPNLEPWNHPGSQPETPCNKCYCKHCSYHCQVCFLTKGLGSNGRKKRRQRRSTPPSSEGHQNPLSKQPLSHTRGDPTGSEESKKKVESKTKADPCD

SEQ ID NO: 9 - protein, 100 aa, Human Immunodeficiency Virus 1
MEPVDPNLEPWKHPGSQPKTACNRCYCKVCCYHCYMCFLKKGLGFYGRKKRKLRRGTPYSSKDHQNPIPKQPLPQAQRDSTGPEKSKKEVESKTEPDRFA

SEQ ID NO: 10 - protein, 100 aa, Human Immunodeficiency Virus 1
MDPVDPNLEPWNQPGSQPKTACNQCYCKRCCYHCQHCFLKKGLGFYGRKKRRQRRTAPYGNKNHQDPIQEQSIPQTQRDSTGSEESKKKVESKARADRFD

SEQ ID NO: 11 - protein, 100 aa, Human Immunodeficiency Virus 1
MEPVDPNLEPWQHPGSQPKTACSNCYCKACCWHCQACFLNKGLGSYGRKKRRQRRGTPQSNKDHQNPVRKQPIPQAQGDSTGPEESKKKVESKTETDQFD

SEQ ID NO: 12 - protein, 100 aa, Human Immunodeficiency Virus 1
MEPVDPNLEPWHHPGSQPKTACNKCYCKACCWHCQVCFLNKGLGSYGRKKRKQRRGTSQNNKDHQNPVPKQPIPQTQGDSTGPEESKKKVESKTETDRFD

SEQ ID NO: 13 - protein, 100 aa, Human Immunodeficiency Virus 1
MDPVDPSLEPWNHPGSQPTTACSKCYCKRCCFHCQKCFLNKGLGSYGRKKRKQRRRTPYSNKDHQNPIPKQPSSQPRGDPTGPEESKKRVESKAETDRLD

SEQ ID NO: 14 - protein, 100 aa, Human Immunodeficiency Virus 1
MDPVDPSLEPWNHPGSQPTTACNKCYCKKCCYHCQSRFLNKGLGSYGRKKRRQRQRTPHSSKDHQNPVPKQPSSQPRGDPTGPKEKKKVEGKAEADRFDW

SEQ ID NO: 15 - protein, 100 aa, Human Immunodeficiency Virus 1
MEPVDPKLEPWKHPGSQPTTACSKCYCKICCWHCQLCFLQKGLGSYGRKKRKHRRRTPQSNKDRQNPIPEQPLSIIRGNPTDPKEQKKKVASKAETDPCD

SEQ ID NO: 16 - protein, 99 aa, Human Immunodeficiency Virus 1
MELVDPNIEPWNQPGSQPTTACSNCYCKICCWHCQLCFLKKGLGSYGRKKRKHRRGTPQSRKDHQNPVPKQPLSITRGNPTDQKESKKEVASKAETDPD

SEQ ID NO: 17 - protein, 100 aa, Human Immunodeficiency Virus 1
MELVDPNLEPWKHPGSKPTTACNNCYCKKCCWHCQICFLKKGLGSYGRKKRRHRRRTPQSSKDHQNPIPEQPPSIIRGNPTGPKESKKEVESKAETDLCD

SEQ ID NO: 18 - protein, 100 aa, Human Immunodeficiency Virus 1
  misc_feature (68)...(68): Xaa is any amino acid
MEPVDPNLEPWNHPGSQPTTACSNCYCKVCCWHCQLCFMTKGLSSYGRKKRKRRRGTPHGSEDHQNLXSKQPSSQPRGDPTGPKEQKKKVESKAEADPFD

SEQ ID NO: 19 - protein, 100 aa, Human Immunodeficiency Virus 1
MEPIDPRLEPWKHPGSQPNTACTPCYCKKCCFHCQVCFLTKGLGSYGRKKRRQRRRPPLDSQNHQASLSKQPTSWSRGDPTGPEESKKKVERETEADPSN

SEQ ID NO: 20 - protein, 100 aa, Human Immunodeficiency Virus 1
MEPVDPRLEPWKHPGSQPRTACTKCYCKKCCLHCQVCFLTKGLGSYGRKKRRQRRRPPQDSQTHQSDLSKQPTAQQRGDPTGPKESKKKVERETETDPDH

SEQ ID NO: 21 - protein, 100 aa, Human Immunodeficiency Virus 1
MEPVDPRLEPWQHPGSQPRTACNKCYCKKCCLHCQVCFMKKGLGSYGRKKRRQRRRPHQDNQNNQVSLSKQPASQSRGNPTGPEESKKKVERETTTDPFA

SEQ ID NO: 22 - protein, 101 aa, Human Immunodeficiency Virus 1
MEPVDPRLEPWKHPGSQPKTACTPCYCKKCCFHCQVCFTKKALGSYGRKKRRQRRRAPEGSQTHQDSLSKQPTSQSRGDPTGPKEPKKKVESETETDPMDR

SEQ ID NO: 23 - protein, 101 aa, Human Immunodeficiency Virus 1
MEPVDPRLEPWKHPGSQPKTACTNCYCKKCCLHCQVCFIKKGLGSYGRKKRRQRRRASQDSQTHQASLSKQPASQPRGDPTGPKESKKKVETETETDPDNQ

SEQ ID NO: 24 - protein, 100 aa, Human Immunodeficiency Virus 1
  misc_feature (5)...(5): Xaa is any amino acid
MEPVXPRLEPWKHPGSQPRTACTNCYCKRCCYHCQXCFITKGLGSYGRKKRRQRRXAXQXSQTHQDSLSXQPASQPRGDPTGPTESKKKVERETETDPLD

SEQ ID NO: 25 - protein, 100 aa, Human Immunodeficiency Virus 1
MEPVDPSLEPWKHPGSQPKTACNNCYCKRCCFHCQKCFTTKGLGSYGRKKRRQRRRASQNSPDHQVSLSKQPASQPQGDPTGPKESKKKVERETETDPVA

SEQ ID NO: 26 - protein, 100 aa, Human Immunodeficiency Virus 1
MEPVDPRLEPWQHPGSQPKTACNNCYCKKCCFHCQKCFTTKGLGSYGRKKRRQRRRAPQDSKTHQSSIPKQPSSQPRGDPTGPKESKEKVERETKADPFD

SEQ ID NO: 27 - protein, 100 aa, Human Immunodeficiency Virus 1
MDPVDPRLEPWNHPGSQPKTACNSCYCKRCCLHCQVCFMTKGLGSYGRKKRGKRRRTPQSGPNHQNIVSKQPSSQPRGDPTGQEEPKKKVEKKTTTDPFD

SEQ ID NO: 28 - protein, 99 aa, Human Immunodeficiency Virus 1
MDPIDPNLEPWNHPGSQPKTPCNNCYCKMCCYHCQFCFTRKGLGYYGRKKRKRRGATESSKDHQDTIPKQPLSQSGGTQTGQKESKKEVEKTTTSDQCA

SEQ ID NO: 29 - protein, 100 aa, Human Immunodeficiency Virus 1
MDPIDPDLEPWKHPGSQPRTVCNNCYCKACCYHCIYCFTKKGLGSYGRKKRTTRRRTAPAGSKNNQDSIPKQPLSQSRGNKEGSEKSTKEVASKTEADQD

(19) United States
(12) Patent Application Publication
(10) Pub. No.: US 2011/0287045 A1
(43) Pub. Date: Nov. 24, 2011

(54) Enhanced immune response against infections

(76) Inventor: Zongdong Li, Rego Park, NY (US)

(21) Appl. No.: 13/068,744

(22) Filed: May 19, 2011

Related U.S. Application Data
(60) Provisional application No. 61/396,006, filed on May 20, 2010.

(51) Int. Cl. A61K 38/16; C07K 14/16; C12N 15/63; A61K 31/7088; A61P 31/04; A61P 31/12; A61P 31/10; A61P 37/04; A61K 39/21; C07H 21/00

(52) U.S. Cl. 424/188.1; 424/281.1; 424/208.1; 530/324; 530/350; 435/320.1; 536/23.72

(57) ABSTRACT

The present invention relates to the discovery that Tat polypeptides may be used as immunoregulators to enhance the immune response to infectious diseases. More particularly, Tat polypeptides may be used as immunoregulators to enhance immune responses to microbial infections, for example, viral and bacterial infections. In accordance with the present invention, Tat polypeptides interact with CCR3 to stimulate platelet activation. The novel finding of the present inventors, therefore, presents new applications for which Tat nucleic and amino acid sequences, and compositions thereof may be used to advantage. Applications for which the Tat nucleic and amino acid sequences and compositions thereof of the invention may be used include, but are not limited to, various research and therapeutic applications as described herein. Also provided is a kit comprising Tat nucleic and/or amino acid sequences, Tat activity compatible buffers, and instruction materials.

### CROSS REFERENCE TO RELATED APPLICATION

This application claims priority under 35 USC §119(e) from U.S. Provisional Application Ser. No. 61/396,006, filed May 20, 2010, which application is herein specifically incorporated by reference in its entirety.

### GOVERNMENT RIGHTS

The research leading to the present invention was funded in part by grants DA020816 and DA004315 from the National Institute of Health. The United States government has certain rights in the invention.

### FIELD OF THE INVENTION

The present invention relates to the field of molecular biology and immunology, and particularly to the discovery of a method for enhancing an immune response. More particularly, the invention relates to a method for enhancing an immune response by achieving heightened platelet activation. Also encompassed are modified transactivating factor (tat) polypeptides and tat peptides that can be used to activate platelets. Methods of use for tat, modified tat polypeptides, and tat peptides are also provided which are of utility in therapeutic applications.

### BACKGROUND OF THE INVENTION

HIV infection not only results in profound host immunosupression, but also immune dys-regulation. For example, HIV-related thrombocytopenia (HIV-1-ITP) was recognized as a manifestation of HIV infection during the AIDS epidemic (Karpatkin. Hematol Oncol Clin North Am. 1990; 4:193-218; Louache et al. Curr Opin Hematol. 1994; 1:369-372; Miguez-Burbano et al. Curr Med Chem Cardiovasc Hematol Agents. 2005; 3:365-376; Scaradavou. Blood Rev. 2002; 16:73-76). It occurs in 15-60% of patients with AIDS, and is seen in about 10% of patients during early stages of HIV infection (Bierling et al. Semin Thromb Hemost. 1995; 21:68-75). To date, the pathogenesis of HIV-1-ITP is not completely understood. It appears that the etiology of HIV-1-ITP in the early and the late stages is different. HIV-1-ITP occurring during the early stages of infection is largely due to immune destruction of platelets by autoantibodies. In its late stages, HIV-1-ITP is likely due to defective thrombopoiesis resulting from HIV-induced impaired bone-marrow production (Bierling et al. Semin Thromb Hemost. 1995; 21:68-75; Najean et al. J Lab Clin Med. 1994; 123:415-420). Early onset HIV-1 ITP is clinically indistinguishable from classic autoimmune idiopathic thrombocytopenia (AITP). However, HIV-1 ITP is different from AITP with respect to markedly elevated platelet-associated IgG, IgM, C3, and C4 and the presence of serum circulating immune complexes (CICs) composed of same. In particular, unlike classic AITP in which multiple antibodies have been described with different specificities for platelet GPIIb, GPIIIa, and GPIb, patients with HIV-1 ITP have an IgG1 antibody against an immunodominant epitope, GPIIIa49-66. The present inventor has previously shown that peptides derived from HIV-1 proteins (Nef, GP120) cross react with an autoantibody against platelet integrin GPIIIa49-66 and thus demonstrated a role of molecular mimicry between HIV-1 proteins and platelet integrin GPIIIa49-66 in inducing anti-GPIIIa49-66 autoantibody (Li et al. Blood. 2005; 106:572-576). The present inventor has also shown that anti-GPIIIa49-66 antibody-induced platelet fragmentation requires a specific conformational change of the GPIIIa integrin (Li et al. J Biol Chem. 2008; 283:3224-3230). While this information has conferred a better understanding regarding the generation of anti-GPIIIa49-66 antibody and how it induces platelet fragmentation, it is still unclear how HIV-1 infection induces the autoimmune response to generate these anti-platelet autoantibodies (Zandman-Goddard et al. Autoimmun Rev. 2002; 1:329-337; Levy. Adv Dent Res. 2006; 19:10-16).

Platelet activation has been reported in HIV-1 infected patients (Ahmad et al. AIDS. 2006; 20:1907-1909; Holme et al. FASEB J. 1998; 12:79-89). Upon activation, platelets not only play a critical role in thrombotic events but are also involved in inflammatory reactions, angiogenesis, and immune responses (Elzey et al. Cell Immunol. 2005; 238:1-9; Elzey et al. Immunity. 2003; 19:9-19). The function of platelets in modulating the immune system is thought to be due to the release of platelet-derived chemokines, cytokines, small chemical molecules, and the expression of a multitude of immune receptors on their membranes.

Among the platelet-derived molecules, CD154 has been investigated intensively due to its important role in regulating B-cell activity. CD154 is a trimeric, transmembrane protein of the tumor necrosis factor family that is present on many different cell types (Schonbeck et al. Cell Mol Life Sci. 2001; 58:4-43). B-cell activation relies on the interaction between CD154 (CD40 ligand) and CD40. Interaction of CD154 with its receptor CD40 on B cells is essential for B-cell proliferation, differentiation, isotype switching, memory B-cell generation, and germinal center formation (Schonbeck et al. Cell Mol Life Sci. 2001; 58:4-43; Elzey et al. J Leukoc Biol. 2005; 78:80-84). It has been suggested that the presence of high levels of CD154 is associated with the development of autoimmune diseases, including autoimmune thrombocytopenia in the absence of HIV disease (Quezada et al. Annu Rev Immunol. 2004; 22:307-328; Toubi et al. Autoimmunity. 2004; 37:457-464; Meabed et al. Hematology. 2007; 12:301-307; Solanilla et al. Blood. 2005; 105:215-218). Blockade of the CD40/CD154 interaction can block the production of autoantibodies and induce the generation of autoantigen-specific anergic CD4 T cells (Toubi et al. Autoimmunity. 2004; 37:457-464; Kuwana et al. Blood. 2004; 103:1229-1236; Kornbluth. J Leukoc Biol. 2000; 68:373-382; Kuwana et al. Blood. 2003; 101:621-623). In addition to CD4⁺ T cells, CD154 is present in platelets (Henn et al. Nature. 1998; 391:591-594). Indeed, studies indicate that 95% of circulating CD154 is derived from platelets (Andre et al. Circulation. 2002; 106:896-899; Henn et al. Blood. 2001; 98:1047-1054).

CD154 is cryptic in resting platelets but is rapidly presented on the platelet surface after platelet activation (Elzey et al. Cell Immunol. 2005; 238:1-9; Henn et al. Nature. 1998; 391:591-594; Sprague et al. Immunol Res. 2007; 39:185-193; von Hundelshausen et al. Circ Res. 2007; 100:27-40). Surface-expressed CD154 is subsequently cleaved over a period of minutes to hours, generating a soluble fragment termed sCD154 that remains trimeric (Henn et al. Blood. 2001; 98:1047-1054). It has been shown that as little as a two fold change of CD154 in serum has a profound effect on B cell activity (Perez-Melgosa et al. J Immunol. 1999; 163:1123-1127). It is therefore conceivable that over-expression or release of platelet associated CD154 may result in an autoimmune response (Quezada et al. Annu Rev Immunol. 2004; 22:307-328; Toubi et al. Autoimmunity. 2004; 37:457-464; Meabed et al. Hematology. 2007; 12:301-307; Solanilla et al. Blood. 2005; 105:215-218). However, the effect of HIV-1 infection on the release of platelet derived CD154 and the role of platelet derived CD154 on B activity have not been examined.

HIV-1 Tat (Trans Activating Factor) encodes a viral regulatory protein with multifunctional activities (Pugliese et al. Cell Biochem Funct. 2005; 23:223-227; Noonan et al. Adv Pharmacol. 2000; 48:229-250). It interacts with a variety of different molecules located inside the cell or on the surface of the cell membrane. The Tat interacts with receptors including CD26, the VEGF receptor, chemokine receptors (CCR1, CCR3, CCR4 and CXCR4), and integrin αVβ3. Tat interacts with integrins through an RGD (Arg-Gly-Asp) motif in its C-terminal domain and interacts with chemokine receptor through CCF/Y, SYXR motifs located between its cysteine-rich region domain and basic domain (Urbinati et al. J Cell Sci. 2005; 118:3949-3958; Urbinati et al. Arterioscler Thromb Vasc Biol. 2005; 25:2315-2320; de Paulis et al. J Immunol. 2000; 165:7171-7179). The chemokine activity of Tat was first identified by noting that Tat is a strong chemoattractant for monocytes. Consequently, a conserved sequence of Tat containing both CC and CXC chemokine motif was identified and it was confirmed that Tat interacts with β-chemokine receptors CCR2 and CCR3. CCR3 is expressed in platelets, mast cells and basophils (Gear et al. Microcirculation. 2003; 10:335-350; Clemetson et al. Blood. 2000; 96:4046-4054). It was reported that Tat interacts with mast cells through CCR3, and may contribute to high IgE level in HIV-1-infected patients (Marone et al. Trends Immunol. 2001; 22:229-232).

The citation of references herein shall not be construed as an admission that such is prior art to the present invention.

Several publications and patent documents are referenced in this application in order to more fully describe the state of the art to which this invention pertains. The disclosure of each of these publications and documents is incorporated by reference herein.

Other features and advantages of the invention will be apparent from the detailed description, the drawings, and the claims.

### SUMMARY OF THE INVENTION

In a first aspect, the present invention relates to the discovery that Tat polypeptides may be used as immunoregulators to enhance the immune response to infectious disease. More particularly, Tat polypeptides may be used as immunoregulators to enhance immune responses to microbial infections, for example, viral and bacterial infections. The invention also extends to functional Tat fragments, variants, and derivatives thereof that act as positive modulators of immune activation responsive to infections, including microbial infections such as viral and bacterial infections. Accordingly, the present invention is directed to methods of using Tat polypeptides and functional fragments, variants, and derivatives thereof as anti-microbial therapeutics. More particularly, the present invention is directed to methods of using Tat polypeptides and functional fragments, variants, and derivatives thereof as anti-viral and/or anti-bacterial drugs.

It is noteworthy that prior to the discovery of the present invention, the chemokine activity of Tat to induce platelet activation was unappreciated. The interaction between Tat and CC chemokine receptor-3 (CCR3) on platelets is a novel discovery and, in accordance with the present invention, manipulating this interaction can be used to enhance or inhibit immune responses. Platelet CCR3 is, therefore, a novel target for the development of therapeutic modulators of immune responses and methods for targeting this interaction are encompassed herein.

It is noteworthy that prior to the discovery of the present invention, the ability of Tat to act as a platelet stimulator was unappreciated. More particularly, the ability of Tat to stimulate platelets to upregulate CD154 expression while inducing minimal and/or unstable platelet aggregation, confers upon Tat polypeptides a unique combination of features that render Tat polypeptides and functional fragments, variants, and derivatives thereof ideally suited as therapeutic molecules for promoting enhanced immune responses to infections.

Accordingly, in a particular embodiment of the invention, Tat polypeptides and functional fragments, variants, and derivatives thereof are used as therapeutic molecules for promoting enhanced immune responses to microbial infections. In a more particular embodiment, Tat polypeptides and functional fragments, variants, and derivatives thereof are used as therapeutic molecules for enhancing immune responses to viral and/or bacterial infections.

The novel finding of the present inventor, therefore, presents new applications for which Tat nucleic and amino acid sequences and compositions thereof may be used to advantage. Such utilities include, but are not limited to, various therapeutic and research applications as described hereinbelow. Also provided is a kit comprising Tat nucleic and/or amino acid sequences, Tat activity compatible buffers, and instruction materials.

Accordingly, in an aspect of the invention, a method is presented for treating a patient with an infectious disease, the method comprising administering to the patient a therapeutically effective amount of a Trans Activating Factor (Tat) polypeptide or a composition comprising a Tat polypeptide, wherein administration of the Tat polypeptide or the composition enhances immune responses to an infectious/etiological agent that causes the infectious disease.

In a particular aspect of the method, the infectious disease is caused by a virus that does not express the Tat polypeptide. Exemplary viruses that do not express Tat polypeptide include, but are not limited to, viral infections caused by hepatitis virus (e.g., hepatitis A, hepatitis B, hepatitis C, hepatitis D, and hepatitis E viruses), herpesviruses (e.g. herpes simplex virus types 1 and 2, varicella-zoster virus, cytomegalovirus, Epstein Barr virus, and human herpes viruses types 6, 7, and 8), and influenza virus. This list is purely illustrative and is in no way to be interpreted as restrictive.

In yet another aspect of the method, the infectious disease is caused by a microbe. In accordance with the present method, a microbe can be a bacteria (Gram positive and Gram negative), fungi, yeast, or mold. Exemplary bacterial infections treatable in accordance with the present method include those caused by Gram-positive cocci, for example Staphylococci (Staph. aureus, Staph. epidermidis) and Streptococci (Strept. agalactiae, Strept. faecalis, Strept. pneumoniae, Strept. pyogenes); Gram-negative cocci (Neisseria gonoirhoeae and Yersinia pestis) and Gram-negative rods such as Enterobacteriaceae, for example Escherichia coli, Hamophilus influenzae, Citrobacter (Citrob. freundii, Citrob. divernis), Salmonella and Shigella, and Francisella (Francisella tularensis); Gram-positive rods such as Bacillus (Bacillus anthracis, Bacillus thuringenesis); furthermore Klebsiella (Klebs. pneumoniae, Klebs. oxytoca), Enterobacter (Ent. aerogenes, Ent. agglomerans), Hafnia, Serratia (Sen. marcescens), Proteus (Pr. mirabilis, Pr. rettgeri, Pr. vulgaris), Providencia, Yersinia, and the genus Acinetobacter. Also encompassed are bacterial infections caused by the genus Pseudomonas (Ps. aeruginosa, Ps. maltophilia) and strictly anaerobic bacteria such as, for example, Bacteroides fragilis, representatives of the genus Peptococcus, Peptostreptococcus and the genus Clostridium; furthermore Mycoplasmas (M. pneumoniae, M. hominis, Ureaplasma urealyticum) as well as Mycobacteria, for example Mycobacterium tuberculosis. This list of microbes is purely illustrative and is in no way to be interpreted as restrictive.

In a particular embodiment, the bacteria is an antibiotic resistant strain. The present method, therefore, encompasses treatment of subjects or patients that have a bacterial infection caused by or associated with at least one antibiotic resistant bacterial strain.

Examples of microbial infections or illnesses that can be treated by administration of the composition of the present invention include, but are not limited to, microbial infections or illnesses in humans such as, for example, otitis, pharyngitis, pneumonia, peritonitis, pyelonephritis, cystitis, endocarditis, systemic infections, bronchitis (acute and chronic), septic infections, illnesses of the upper airways, diffuse panbronchiolitis, pulmonary emphysema, dysentery, enteritis, liver abscesses, urethritis, prostatitis, epididymitis, gastrointestinal infections, bone and joint infections, skin infections, postoperative wound infections, abscesses, phlegmon, wound infections, infected burns, burns, infections in the mouth (including, e.g., but not limited to, periodontal disease and gingivitis), infections after dental operations, osteomyelitis, septic arthritis, cholecystitis, peritonitis with appendicitis, cholangitis, intraabdominal abscesses, sinusitis, mastoiditis, mastitis, tonsileitis, typhoid, meningitis and infections of the nervous system, salpingitis, endometritis, genital infections, pelveoperitonitis and eye infections.

In yet another aspect of the invention, the method calls for administration of a Tat polypeptide that comprises the amino acid sequence of SEQ ID NO: 2 [Full length (FL) Tat is 101 amino acids (aa); see FIG. 12]. Additional variants and truncations of SEQ ID NO: 2 are depicted in FIG. 12 and designated with respect to SEQ ID NO: 2 as: A: 22-101 aa of FL; B: 1-21 aa linked to 38-101 aa; C: 1-37 aa linked to 49-101 aa; D: 1-48 aa linked to 58-101 aa; E: 1-57 aa linked to 72-101 aa; F: 1-77 aa linked to 81-101 aa; and G: 1-72 aa. Additional Tat variants that may be used in the methods of the invention are shown in FIG. 13.

Although not wishing to be bound by theory, Tat variants and/or truncations comprising a CCR3 binding motif and the RGD motif are envisioned as exemplary Tat polypeptides having the ability to induce platelet activation and CD154 release.

In other embodiments, Tat variants and/or truncations described herein may be used as inhibitors of CCR3 or beta3 integrin activity.

In an embodiment of the invention, the method further comprises administering an anti-viral agent to the patient. Exemplary anti-viral agents for use in this combinatorial context include, for example, acyclovir, valacyclovir, famciclovir, ganciclovir, amantadine, and rimantadine. Anti-viral agents can be administered before, concurrently, or after the Tat polypeptides of the invention. Anti-viral agents can be administered concurrently in separate formulations or can be administered in a composition comprising both a Tat polypeptide and an anti-viral agent.

In another embodiment of the invention, the method further comprises administering an anti-bacterial agent to the patient. Exemplary anti-bacterial agents for use in this combinatorial context include, for example, antibiotics. Antibiotics for use in combinatorial applications include, without limitation, amoxicillin (a type of penicillin); fluoroquinolones such as ciprofloxacin (Cipro) and trovafloxacin (Trovan); doxycycline; Bactrim (trimethoprim-sulfamethoxazole); fluconazole; cefazolin (Ancef, Kefzol); cefamandole (Mandol); cefotaxime (Claforan); and cefoxitin (Mefoxin). Anti-bacterial agents can be administered before, concurrently, or after the Tat polypeptides of the invention. Anti-bacterial agents can be administered concurrently in separate formulations or can be administered in a composition comprising both a Tat polypeptide and an anti-bacterial agent.

Also provided is a composition comprising at least one Tat polypeptide, including a functional variant, derivative, or fragment of SEQ ID NO: 2, and a pharmaceutically acceptable buffer. Compositions comprising a nucleic acid sequence encoding a Tat polypeptide, including a functional variant, derivative, or fragment of SEQ ID NO: 2, and a pharmaceutically acceptable buffer are also envisioned. Additional Tat variants are shown in FIGS. 12 and 13.

The invention also encompasses a composition comprising a Tat polypeptide and an anti-viral or an anti-bacterial agent. In a particular embodiment, a composition comprising a Tat polypeptide and an anti-viral that is designed to treat a non-Tat expressing virus infection or is specific for a non-Tat expressing virus infection is envisioned. In a more particular embodiment, such anti-virals include those agents which exhibit little or no anti-viral activity with respect to Tat expressing viruses.

In an aspect of the invention, a method is presented for treating a patient with a disorder, the method comprising administering to the patient a therapeutically effective amount of a composition of the invention to alleviate symptoms of the disorder. The composition comprises at least one Tat polypeptide of the invention capable of promoting immune responses in the patient to alleviate symptoms of the disorder in the patient, which symptoms depend on the disorder afflicting the patient.

The methods of the invention are particularly useful for the treatment of an infectious disease, wherein administering a therapeutically effective amount of a composition of the invention comprising at least one Tat polypeptide capable of augmenting immune responsiveness to a patient alleviates symptoms of the infectious disease by reducing the number of infectious units in the patient. Infectious diseases treatable using the compositions and methods of the invention include, but are not limited to, bacterial and viral infections. In a particular embodiment, administration of a therapeutically effective amount of a composition of the invention alleviates symptoms of the bacterial or viral infection by reducing the number of bacteria or viruses in the patient.

In another embodiment of the invention, the method relates to identification of a patient afflicted with a bacterial infection and administering a therapeutically effective amount of a Tat polypeptide or variant, derivative, or functional fragment thereof to the patient to treat the patient, thereby alleviating symptoms associated with the bacterial infection.

In yet another embodiment of the invention, the method relates to identification of a patient afflicted with a viral infection caused by a virus that does not express a Tat polypeptide and administering a therapeutically effective amount of a Tat polypeptide or variant, derivative, or functional fragment thereof to the patient to treat the patient, thereby alleviating symptoms associated with the viral infection.

In yet another aspect of the invention, the method relates to using Tat polypeptides to treat platelets ex vivo to generate activated platelets for re-introduction into a patient. In accordance with the invention, the platelets may be isolated from a patient, treated ex vivo with Tat polypeptides, and reintroduced into the patient of origin. Such transfusions are referred to herein as autologous. Alternatively, platelets may be isolated from a donor (different subject or subjects), treated ex vivo with Tat polypeptides and introduced into a recipient patient. Such transfusions are referred to herein as syngeneic (wherein the donor is an identical twin) or allogeneic. In particular embodiments, the recipient is a cancer patient in need of a platelet transfusion following chemotherapy or a patient infected with a virus or bacteria.

Ex vivo activated platelets are activated to release CD154 as well as other cytokines. Release of platelet-derived microparticles (PMPs) from such ex vivo activated platelets, moreover, leads to presentation of CD154 trimers, which are competent to engage CD40 on B cells. Such engagement, in turn, leads to B cell proliferation, differentiation, isotype switching, memory B-cell generation, and germinal center formation.

Also encompassed by the present invention are methods for using a CCR3 antagonist or agonist to modulate platelet activation. In accordance with the invention, a method is described wherein a CCR3 antagonist is used to inhibit platelet activation. Such methods may be performed in vitro or in vivo. CCR3 antagonists are known in the art and include, without limitation, SB-297006; SB-328437; GW701897B; YM-344031; DPC168 (a benzylpiperidine-substituted aryl urea CCR3 antagonist) and its acetylpiperidine derivative, BMS-570520; BMS-639623; and pyrazolone methylamino piperidine derivatives. See, for example, Erin et al. (2002, Curr Drug Targets Inflamm Allergy 1:201-214), Pruitt et al. (2007, Bioorganic & Medicinal Chemistry Letters, 17: 2992-2997), Santella III et al. (2008, Bioorganic & Medicinal Chemistry Letters, Volume 18:576-585), Pégurier et al. (2007, Bioorganic & Medicinal Chemistry Letters, Volume 17:4228-4231), and Wacker et al. (2002, Bioorganic & Medicinal Chemistry Letters, Volume 12:1785-1789), which are incorporated herein in their entireties. In a further embodiment, a method is described wherein a CCR3 agonist is used to promote platelet activation. CCR3 agonists are known in the art and include, without limitation, TY-18526 and CH0076989. See, for example, Masayuki et al. (2004, Allergy in Practice 322:792-795), Wise et al. (2007, J Biol Chem 282:27935-27943), and Ting et al. (2005, Bioorganic and Medicinal Chem Letters 15:3020-3023), which are incorporated herein in their entireties.

In another aspect of the invention, an isolated amino acid sequence comprising a polypeptide having the amino acid sequence of SEQ ID NO: 2 or having sequence and/or structural homology to SEQ ID NO: 2, or a functional variant, derivative, or fragment of SEQ ID NO: 2, wherein said polypeptide is capable of promoting platelet activation is presented. Also included are expression vectors encoding an amino acid sequence of the invention, wherein expression of the amino acid sequence is controlled by regulatory sequences in the expression vector and cells comprising such expression vectors.

In another aspect of the invention, an isolated nucleic acid sequence encoding an amino acid sequence comprising SEQ ID NO: 2 or a variant thereof, wherein the amino acid sequence is a Tat polypeptide or a functional variant, derivative, or fragment thereof capable of promoting platelet activation is presented. Additional Tat variants are shown in FIGS. 12 and 13. In a particular embodiment, the isolated nucleic acid sequence encoding an amino acid sequence comprising SEQ ID NO: 2 is SEQ ID NO: 1. See FIG. 14.

Also provided is an expression vector comprising an isolated nucleic acid sequence encoding an amino acid sequence comprising SEQ ID NO: 2 or a variant thereof, wherein the amino acid sequence is a Tat polypeptide or a functional variant, derivative, or fragment thereof capable of promoting platelet activation, and wherein the isolated nucleic acid encoding an amino acid sequence comprising SEQ ID NO: 2 or a variant thereof is operably linked to a regulatory sequence. Cells comprising these expression vectors are also envisioned. Such expression vectors and cells comprising same, as well as compositions comprising such expression vectors and cells, as described herein and above, are envisioned as useful agents for treating a patient with an infectious disease to promote enhanced immune responses to the infectious/etiological agent that is causing or is associated with the infectious disease.

In a particular embodiment, an expression vector is provided that comprises the nucleic acid sequence of SEQ ID NO: 1 or a variant, derivative, or fragment thereof, wherein the nucleic acid sequence encodes a Tat polypeptide capable of promoting platelet activation, and SEQ ID NO: 1 or the variant, derivative, or fragment thereof is operably linked to a regulatory sequence. Cells comprising such expression vectors are also presented envisioned. Such expression vectors and cells comprising same, as well as compositions comprising such expression vectors and cells, as described herein and above, are envisioned as useful agents for treating a patient with an infectious disease to promote enhanced immune responses to the infectious/etiological agent that is causing or is associated with the infectious disease.

In a further aspect of the invention, a kit comprising Tat nucleic and/or amino acid sequences, Tat activity compatible buffers, and instruction materials is provided.

### BRIEF DESCRIPTION OF THE DRAWINGS

FIG. 1(a)-(i) show that Tat induces human platelet activation. Histogram of flow cytometry analysis of the platelets treated with Tat or Ad18-66 (Ctrl, filled histogram) with CD154 (a) or CD62P (b). Histogram is representative of one of three experiments. Bar graph summary of Tat induced platelet activation and CD154 expression (c). Both Tat and CCL5 induce mouse platelet activation. Histogram of flow cytometry analysis of the platelets treated with Tat, CCL5 or Ad18-66 (Ctrl, filled histogram). Histogram is representative of one of three experiments. Summary of Tat and CCL5 induced mouse platelet activation (f). Tat interacts with platelets through integrin β3. Platelets incubated with S³⁵-Tat or S³⁵-luciferase in PBS for 30 minutes at 37° C., washed and lysed for total protein were resolved on a SDS-PAGE gel (g). Western blot analysis for β3 with GST-Tat or GST pulls down protein from platelet lysates (h). Western blot analysis of CD154 release in supernatant of platelets incubated with Tat or with PBS buffer as control (i).

FIG. 2(a)-(f) reveal that Tat-induced platelet activation is integrin β3 dependent. Histogram of flow cytometry analysis of platelets treated with Tat, CCL5, Thrombin and Ad18-66 (Ctrl, filled histogram) with CD154 (a) or CD62P (b). Histogram is representative of one of three experiments. Platelets from integrin β3 knockout mice were employed. Summary of flow cytometry results of Tat with integrin β3 knock out platelets (c, d). Histogram of flow cytometry analyses of platelets for CD154 (e) or CD62P (f) from integrin β3 knockout mice compared with platelets from wild type mice, both were treated with Tat.

FIG. 3(a)-(d) depict the effect of various treatments on platelet activation. Histograms of flow cytometry analysis of platelets treated with Tat, CCL5 and control as follows: (a) with Ad18-66 (Ctrl; n=6); (b) with CCL5, with or without CCR3 inhibitor SB328437 (n=6); (c) with Tat, with or without PGE1 compared (n=6); (d) with Tat, with or without Calcium flux inhibitor EGTA (n=3).

FIG. 4(a)-(e) show that Tat expression cells induce platelet activation in vivo. Western Blot to detect the Tat expression in TAT-4T1 cells (a). RT-PCR of Tat with RNA from TAT-4T1 cells. Specific Tat band 359 bp was found with the MSCV-TAT cells (left lane), MSCV cells (right lane) (b). Western Blot to detect the Tat expression in serum of mouse injected with TAT-MSCV (c). Summary of flow cytometry analysis of CD154 expression (d) and platelet activation (CD62P) (e) induced by TAT-MSCV retrovirus or by TAT-4T1 cell line injection (n=3).

FIG. 5(a)-(h) demonstrate that Tat activated platelets enhance the B cell activity in vitro. An example of flow cytometry analysis of splenic B cells co-cultured with platelets with Tat or Ad18-66 (Ctrl). The population with high expression of CD45 was gated and the percentages with positive IgG1 (a,b,c) and IgG2b (d,e,f) are shown. Bar-graph summary of flow data for average fluorescent intensity (g) and percentage of positive cells (h) (n=6, p<0.05 for both).

FIG. 6(a)-(g) show that Tat activated platelets enhance B cell activity in vivo. Bar-graph summary of flow cytometry result of positive IgG1 and IgG2b cells in peripheral blood from TAT expression 4T1 cells injected BALB/c mice for average fluorescent intensity (a) and percentage of positive cells (b) (n=6, p<0.05 for both). ELISA results of IgG1 (c) and IgG2b (d) in serum from TAT-MSCV virus and TAT-4T1 cells injected BALB/c mice (n=3). Histogram of flow cytometry analysis of splenic B cells from BALB/c mice injected with Tat or Ad18-66 (Ctrl) for IgG1 (e) and IgG2b (f). Enhanced immune response was found in mice treated with Tat. BALB/c mice were injected with Tat or Ad18-66 (Ctrl) protein through intravenous injection before immunization with Adenovirus. The generation of antibody against Adenovirus was monitored by ELISA (n=3) (g).

FIG. 7 depicts electron microscopy of platelets treated with Tat or control. Left upper panel: Control, low magnification; Right upper panel: Tat, low magnification. Left bottom panel: Control, high magnification. Right bottom panel: Tat, high magnification. The release of platelet-derived micro-particles (PMPs) (upper right) and the PMP buds at the plasma membrane of Tat activated platelets (bottom right) are indicated by arrow.

FIG. 8 shows a model of the role of HIV-1 induced platelet activation in HIV-1 ITP. Tat is expressed by HIV-1 infected cells and activates platelets through chemokine receptor CCR3 and integrin β3. The activated platelets release CD154 and other chemokines and cytokines. CD154 enhances B cell activity and contributes to autoimmune response to platelet. Platelet derived chemokines and cytokines may also affect the megakaryopoiesis.

FIG. 9(a)-(b) shows that integrin β3 deficency and CCR3 inhibitor SB328437 do not inhibit thrombin induced platelet activation. Histogram of flow cytometry analysis thrombin induced platelet activation in β3−/− platelets pre-treated CCR3 inhibitor SB328437 and control (nil). (n=3) (a). Summary of flow cytometry result of thrombin induced platelet activation in β3−/− platelets pre-treated CCR3 inhibitor SB328437 and control (nil) (b).

FIG. 10(a)-(b) reveals that EGTA completely blocks CCL5 induced platelet activation while prostaglandin E1 (PGE1) partially blocks CCL5 induced platelet activation. Flow cytometry analysis of platelets treated with CCL5 with or without PGE1 or EGTA. Control (nil) n=3. Histogram of flow cytometry analysis CD154 (a) and CD62P (b).

FIG. 11(a)-(b) depicts Tat induced microparticle release from platelets. Dot plot of flow cytometry analysis of platelets incubated with control (buffer), Tat 0.5 μg/ml, thrombin (1 U/ml), collagen (1 mg/ml) 37° C. one hour. (a) Dot plot of gated microparticles staining with CD41 and Annexin V (AnnV), percentages of double positive mircoparticles are shown. (b) Dot plot of platelets and microparticles gated with CD41. Percentages of microparticles are shown.

FIG. 12 shows the amino acid sequence of a Tat polypeptide (SEQ ID NO: 2) and a schematic of derivatives thereof.

FIG. 13 shows an alignment of Tat variants comprising domains important for platelet activation. SEQ ID NOs: 3-29 are depicted therein as listed from top to bottom.

FIG. 14 shows a nucleic acid sequence (SEQ ID NO: 1) that encodes SEQ ID NO: 2.

### DETAILED DESCRIPTION OF THE INVENTION

Before the present discovery and methods of use thereof are described, it is to be understood that this invention is not limited to particular assay methods, or test compounds and experimental conditions described, as such methods and compounds may vary. It is also to be understood that the terminology used herein is for the purpose of describing particular embodiments only, and is not intended to be limiting, since the scope of the present invention will be limited only the appended claims.

Platelets play an important role in the innate and adaptive immune response. This is achieved by the regulated expression of adhesive inflammatory mediators and cytokines, which can mediate the interaction with leukocytes and enhance their recruitment. In addition, platelets are characterized by an enormous surface area and open canalicular system, which in concert with specialized recognition receptors may contribute to the engulfment of serum components, antigens, and pathogens.

Platelet derived CD154 has been shown to induce dendritic cell maturation, B cell isotype switching and CD8 T-cell response in vitro and in vivo. Platelet transfusion studies have demonstrated that platelet-derived CD154 is sufficient to induce isotype switching and augment T lymphocyte function during viral infection, leading to enhanced protection against the viral rechallenge. Depletion of platelets in normal mice resulted in decreased antigen-specific antibody production.

As described herein, the present inventor has investigated the effect of HIV-1 Tat protein on platelet associated CD154 expression. Tat (Trans Activating Factor), is a regulatory protein of HIV which is indispensable for viral replication. Its constitution varies from 86 to 104 amino acids (AA) with a molecular weight of 14-16 kDa, due to alternative splicing of mRNA. Incomplete forms of this viral protein (from 58 to 72 AA) are also biologically active. Although Tat protein accumulates in the nucleus of HIV-infected cells, it can also act as a pleiotropic exogeneous factor for bystander cells, due to its ability to induce various biological effects in different cellular types. It contains an RGD motif (a universal binding site for integrins) at its C terminal end and interacts with alphaVbeta3 integrin on endothelial cells. Since the expression and release of platelet CD154 is regulated by integrin alpha2bbeta3, the present inventor sought to examine whether Tat can regulate platelet associated CD154 expression through integrin GPIIbIIIIa.

As described herein, the present inventor has investigated the interaction between Tat and platelets and demonstrated that: 1) Tat is able to induce platelet activation and the release of platelet CD154 and microparticles; 2) Tat-induced platelet activation requires the chemokine receptor CCR3 and β3 integrin; 3) Tat-induced platelet activation is cAMP independent and calcium flux dependent; 4) Tat expression cell line 4T1 and TAT-MSCV retrovirus induce platelet activation in vivo. 5) platelet derived CD154 stimulates B cell activity by enhancing Ig class switching; and 6) over-expression of Tat is able to induce platelet activation in vivo.

In light of the above, the results presented herein suggest that Tat-induced platelet CD154 may play a role in and contribute to the development of HIV-1 ITP.

Intriguingly, although many platelets stimulators can induce the expression of CD154, Tat can also modulate platelet aggregation through its RGD motif binding to alpha2bbeta3. In keeping with the above, the present inventor has not observed any significant platelet aggregation induced by Tat in the instant experiments.

In order to more clearly set forth the parameters of the present invention, the following definitions are used:

As used in this specification and the appended claims, the singular forms “a”, “an”, and “the” include plural references unless the context clearly dictates otherwise. Thus for example, reference to “the method” includes one or more methods, and/or steps of the type described herein and/or which will become apparent to those persons skilled in the art upon reading this disclosure and so forth.

The term “complementary” refers to two DNA strands that exhibit substantial normal base pairing characteristics. Complementary DNA may, however, contain one or more mismatches.

The term “hybridization” refers to the hydrogen bonding that occurs between two complementary DNA strands.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

The phrase “flanking nucleic acid sequences” refers to those contiguous nucleic acid sequences that are 5′ and 3′ to a particular nucleic acid or nucleic acid recognition site.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it is generally associated in its natural state (i.e., in cells or tissues). An isolated nucleic acid (either DNA or RNA) may further represent a molecule produced directly by biological or synthetic means and separated from other components present during its production.

“Natural allelic variants”, “mutants” and “derivatives” of particular sequences of nucleic acids refer to nucleic acid sequences that are closely related to a particular sequence but which may possess, either naturally or by design, changes in sequence or structure. By closely related, it is meant that at least about 60%, but often, more than 85%, 90%, 95%, 97%, 98%, or 99% of the nucleotides of the sequence match over the defined length of the nucleic acid sequence referred to using a specific SEQ ID NO. Changes or differences in nucleotide sequence between closely related nucleic acid sequences may represent nucleotide changes in the sequence that arise during the course of normal replication or duplication in nature of the particular nucleic acid sequence. Other changes may be specifically designed and introduced into the sequence for specific purposes, such as to change an amino acid codon or sequence in a regulatory region of the nucleic acid. Such specific changes may be made in vitro using a variety of mutagenesis techniques or produced in a host organism placed under particular selection conditions that induce or select for the changes. Such sequence variants generated specifically may be referred to as “mutants” or “derivatives” of the original sequence.

The terms “percent similarity”, “percent identity” and “percent homology” when referring to a particular sequence are used as set forth in the University of Wisconsin GCG software program and are known in the art.

The present invention also includes active portions, fragments, derivatives and functional mimetics of Tat polypeptides or proteins of the invention. An “active portion” of a Tat polypeptide means a peptide that is less than the full length Tat polypeptide, but which retains measurable biological activity. With respect to the ability of Tat polypeptides to induce platelet activation and CD154 release, exemplary active portions comprise the CCR3 binding motif and the RGD motif alone, and in combination.

A “fragment” or “portion” of the Tat polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. A “derivative” of the Tat polypeptide or a fragment thereof means a polypeptide modified by varying the amino acid sequence of the protein, e.g. by manipulation of the nucleic acid encoding the protein or by altering the protein itself. Such derivatives of the natural amino acid sequence may involve insertion, addition, deletion or substitution of one or more amino acids, and may or may not alter the essential activity of the original Tat polypeptide.

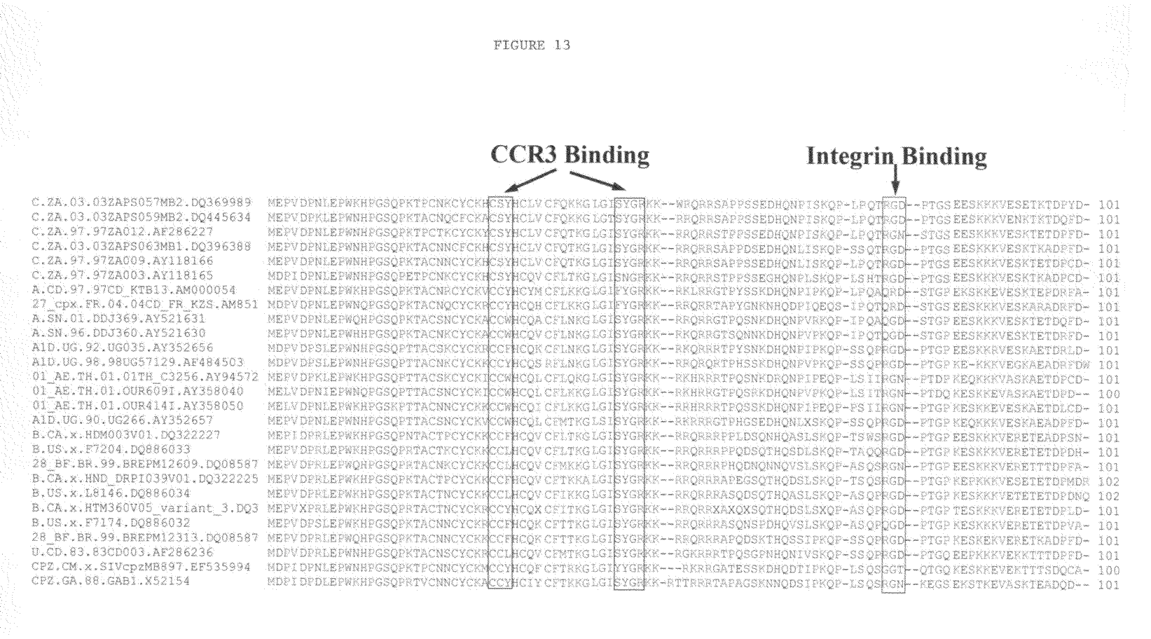

Different “variants” of the Tat polypeptide exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acids residues are substituted with conservative or non-conservative amino acids, (b) variants in which one or more amino acids are added to the Tat polypeptide, (c) variants in which one or more amino acids include a substituent group, and (d) variants in which the Tat polypeptide is fused with another peptide or polypeptide such as a fusion partner, a protein tag or other chemical moiety, that may confer useful properties to the Tat polypeptide, such as, for example, an epitope for an antibody, a polyhistidine sequence, a biotin moiety and the like. Other Tat polypeptides of the invention include variants in which amino acid residues from one viral variant are substituted for the corresponding residue in another variant, either at conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to a person having ordinary skill in the art. The amino acid sequences of exemplary Tat variants are presented in FIG. 13.

To the extent such analogues, fragments, derivatives, mutants, and modifications, including alternative nucleic acid processing forms and alternative post-translational modification forms result in derivatives of the Tat polypeptide that retain any of the biological properties of the Tat polypeptide, they are included within the scope of this invention.

The term “functional” as used herein implies that the nucleic or amino acid sequence is functional for the recited assay or purpose.

The term “functional fragment” as used herein implies that the nucleic or amino acid sequence is a portion or subdomain of a full length polypeptide and is functional for the recited assay or purpose.

The phrase “consisting essentially of” when referring to a particular nucleotide or amino acid means a sequence having the properties of a given SEQ ID NO:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression vector” or “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the term “operably linked” refers to a regulatory sequence capable of mediating the expression of a coding sequence and which are placed in a DNA molecule (e.g., an expression vector) in an appropriate position relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Primers may be labeled fluorescently with 6-carboxyfluorescein (6-FAM). Alternatively primers may be labeled with 4,7,2′,7′-Tetrachloro-6-carboxyfluorescein (TET). Other alternative DNA labeling methods are known in the art and are contemplated to be within the scope of the invention.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity of the isolated polypeptide, and that may be present, for example, due to incomplete purification, addition of stabilizers, or compounding into, for example, immunogenic preparations or pharmaceutically acceptable preparations.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like). “Mature protein” or “mature polypeptide” shall mean a polypeptide possessing the sequence of the polypeptide after any processing events that normally occur to the polypeptide during the course of its genesis, such as proteolytic processing from a polypeptide precursor. In designating the sequence or boundaries of a mature protein, the first amino acid of the mature protein sequence is designated as amino acid residue 1.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties to the sequence, particularly with regard to methods relating to the detection or isolation of the sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other applications, the introduced nucleic acid may exist in the recipient cell or host organism only transiently.

A “clone” or “clonal cell population” is a population of cells derived from a single cell or common ancestor by mitosis.

A “cell line” is a clone of a primary cell or cell population that is capable of stable growth in vitro for many generations.

The compositions containing the molecules or compounds of the invention can be administered for pharmaceutical or therapeutic purposes. In pharmaceutical or therapeutic applications, compositions are administered to a patient suffering from an infectious disease (such as, e.g., a disease caused by a bacterial or viral infection) in an amount sufficient to cure or at least partially arrest the symptoms of the disease and its complications. An amount adequate to accomplish this is defined as a “therapeutically effective amount or dose.” Amounts effective for this use will depend on the severity of the disease and the weight and general state of the patient.

With respect to an infectious disease caused by a bacterial or viral infection, the compositions and methods of the invention can be used to advantage to enhance immune responses to the bacterial or viral infection. It is to be understood that the method and compositions of the invention can be used to enhance immune responses in a patient infected with a single type of bacteria and/or virus or a plurality of types of different bacteria and/or viruses.

Bacterial infections that can be treated using the methods and compositions of the invention include those caused by Gram-positive cocci, for example Staphylococci (Staph. aureus, Staph. epidermidis) and Streptococci (Strept. agalactiae, Strept. faecalis, Strept. pneumoniae, Strept. pyogenes); Gram-negative cocci (Neisseria gonoirhoeae and Yersinia pestis) and Gram-negative rods such as Enterobacteriaceae, for example Escherichia coli, Hamophilus influenzae, Citrobacter (Citrob. freundii, Citrob. divernis), Salmonella and Shigella, and Francisella (Francisella tularensis); Gram-positive rods such as Bacillus (Bacillus anthracis, Bacillus thuringenesis); furthermore Klebsiella (Klebs. pneumoniae, Klebs. oxytoca), Enterobacter (Ent. aerogenes, Ent. agglomerans), Hafnia, Serratia (Serr. marcescens), Proteus (Pr. mirabilis, Pr. rettgeri, Pr. vulgaris), Providencia, Yersinia, and the genus Acinetobacter. Also encompassed are bacterial infections caused by the genus Pseudomonas (Ps. aeruginosa, Ps. maltophilia) and strictly anaerobic bacteria such as, for example, Bacteroides fragilis, representatives of the genus Peptococcus, Peptostreptococcus and the genus Clostridium; furthermore Mycoplasmas (M. pneumoniae, M. hominis, Ureaplasma urealyticum) as well as Mycobacteria, for example Mycobacterium tuberculosis.

Viral infections that can be treated using the methods and compositions of the invention include those caused by hepatitis virus (e.g., hepatitis A, hepatitis B, hepatitis C, hepatitis D, and hepatitis E viruses), herpesviruses (e.g. herpes simplex virus types 1 and 2, varicella-zoster virus, cytomegalovirus, Epstein Barr virus, and human herpes viruses types 6, 7, and 8), and influenza virus. With respect to genuses of viruses and particular viruses, the present method is directed to those viruses that does not express the Tat polypeptide. One of ordinary skill in the art would appreciate which viruses express Tat polypeptide and which do not. Moreover, not all Tat polypeptides include the CCR3 binding sites and integrin binding site important for platelet activation. See FIG. 13.

An “immune response” signifies any reaction produced by an antigen, such as a protein antigen, in a host having a functioning immune system. Immune responses may be either humoral, involving production of immunoglobulins or antibodies, or cellular, involving various types of B and T lymphocytes, dendritic cells, macrophages, antigen presenting cells and the like, or both. Immune responses may also involve the production or elaboration of various effector molecules such as cytokines, lymphokines, chemokines, and the like. Immune responses may be measured both in in vitro and in various cellular or animal systems.

As used herein, the ability to “enhance immune responses” refers to the ability of a molecule (e.g., a Tat polypeptide or functional variant, derivative, or fragment thereof) to promote or augment an immune response.

As used herein, the term “platelet activation” refers to the ability of a molecule (e.g., a Tat polypeptide or functional variant, derivative, or fragment thereof) to induce CD154 release from platelets.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunloglobulin molecule such as those portions known in the art as Fab, Fab′, F(ab′)2 and F(v).

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and described the methods and/or materials in connection with which the publications are cited.

### I. Preparation of Tat-Encoding Nucleic Acid Molecules and Tat Proteins

### A. Nucleic Acid Molecules

Nucleic acid molecules encoding the Tat polypeptides of the invention may be prepared by two general methods: (1) Synthesis from appropriate nucleotide triphosphates; or (2) Isolation from biological sources. Both methods utilize protocols well known in the art.

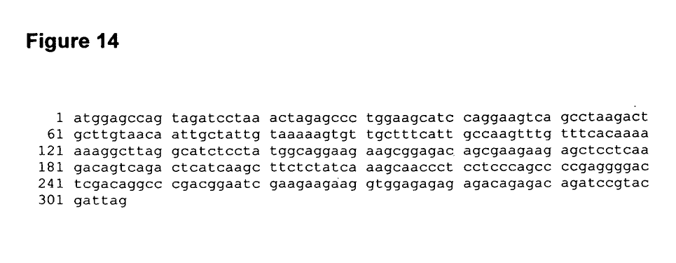

The availability of nucleotide sequence information, such as the full length Tat gene having SEQ ID NO: 1 (See FIG. 14), enables preparation of an isolated nucleic acid molecule of the invention by oligonucleotide synthesis. Synthetic oligonucleotides may be prepared by the phosphoramidite method employed in the Applied Biosystems 380A DNA Synthesizer or similar devices. The resultant construct may be purified according to methods known in the art, such as high performance liquid chromatography (HPLC). Long, double-stranded polynucleotides, such as a DNA molecule of the present invention, must be synthesized in stages, due to the size limitations inherent in current oligonucleotide synthetic methods. Synthetic DNA molecule constructed by such means may then be cloned and amplified in an appropriate vector. Nucleic acid sequences encoding Tat polypeptide may be isolated from appropriate biological sources using methods known in the art. In a preferred embodiment, a full length Tat gene is isolated from an expression library of bacterial origin. In an alternative embodiment, viral variants encoding Tat may be isolated utilizing the sequence information provided by SEQ ID NO: 1.

In accordance with the present invention, nucleic acids having the appropriate level of sequence homology with the protein coding region of SEQ ID NO: 1 may be identified by using hybridization and washing conditions of appropriate stringency. For example, hybridizations may be performed using a hybridization solution comprising: 5×SSC, 5× Denhardt's reagent, 0.5-1.0% SDS, 100 micrograms/ml denatured, fragmented salmon sperm DNA, 0.05% sodium pyrophosphate and up to 50% formamide. Hybridization is generally performed at 37-42° C. for at least six hours. Following hybridization, filters are washed as follows: (1) 5 minutes at room temperature in 2×SSC and 0.5-1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology is (Sambrook et al., 1989):

Tₘ=81.5° C. 16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

As can be seen from the above, the stringency of the hybridization and wash depend primarily on the salt concentration and temperature of the solutions. In general, to maximize the rate of annealing of the two nucleic acid molecules, the hybridization is usually carried out at 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 micrograms/nil denatured salmon sperm DNA at 42° C. and wash in 2×SSC and 0.5% SDS at 55° C. for 15 minutes. A high stringency hybridization is defined as hybridization in 6×SSC, 5× Denhardt's solution, 0.5% SDS and 100 micrograms/ml denatured salmon sperm DNA at 42° C. and wash in 1×SSC and 0.5% SDS at 65° C. for 15 minutes. A very high stringency hybridization is defined as hybridization in 6×SSC, 5× Denhardt's solution, 0.5% SDS and 100 micrograms/ml denatured salmon sperm DNA at 42° C. and wash in 0.1×SSC and 0.5% SDS at 65° C. for 15 minutes.

Nucleic acids of the present invention may be maintained as DNA in any convenient cloning vector. In a preferred embodiment, clones are maintained in a plasmid cloning/expression vector, such as pBluescript (Stratagene, La Jolla, Calif.), which is propagated in a suitable E. coli host cell.

Tat-encoding nucleic acid molecules of the invention include cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule of the present invention, such as selected segments of SEQ ID NO: 1. Such oligonucleotides are useful as probes for detecting or isolating Tat genes.

It will be appreciated by persons skilled in the art that variants of these sequences exist in viral populations, and must be taken into account when designing and/or utilizing oligonucleotides of the invention. Accordingly, it is within the scope of the present invention to encompass such variants, with respect to the Tat sequences disclosed herein or the oligonucleotides targeted to specific locations on the respective genes or RNA transcripts. With respect to the inclusion of such variants, the term “natural allelic variants” is used herein to refer to various specific nucleotide sequences and variants thereof that would occur in a given DNA population. Genetic polymorphisms giving rise to conservative or neutral amino acid substitutions in the encoded protein are examples of such variants. Additionally, the term “substantially complementary” refers to oligonucleotide sequences that may not be perfectly matched to a target sequence, but the mismatches do not materially affect the ability of the oligonucleotide to hybridize with its target sequence under the conditions described.

Thus, the coding sequence may be that shown in SEQ ID NO: 1, or it may be a mutant, variant, derivative or allele of this sequence. The sequence may differ from that shown by a change which is one or more of addition, insertion, deletion and substitution of one or more nucleotides of the sequence shown. Changes to a nucleotide sequence may result in an amino acid change at the protein level, or not, as determined by the genetic code.

Thus, nucleic acid according to the present invention may include a sequence different from the sequence shown in SEQ ID NO: 1 but which encodes a polypeptide with the same amino acid sequence.

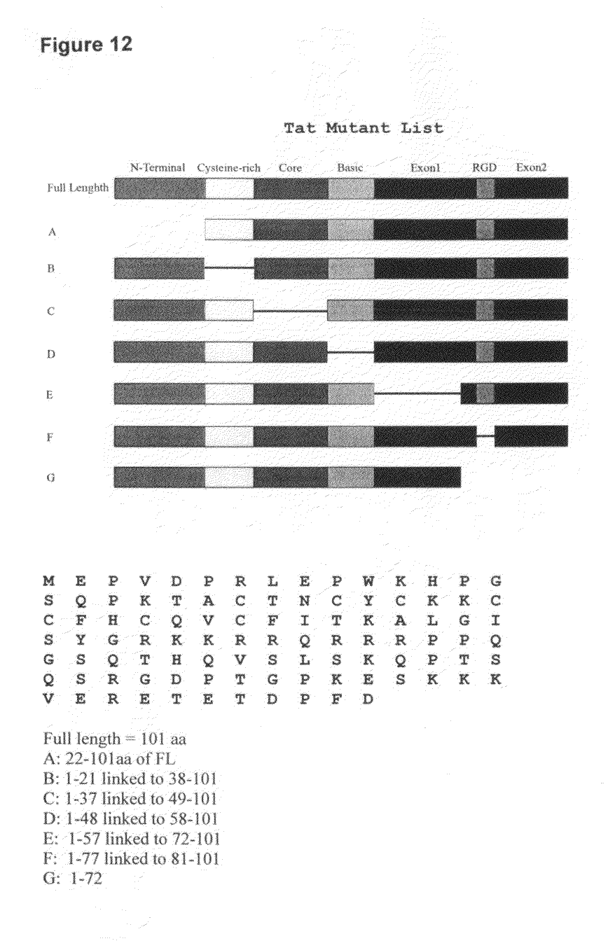

On the other hand, the encoded polypeptide may comprise an amino acid sequence which differs by one or more amino acid residues from the amino acid sequence shown in SEQ ID NO: 2. See FIGS. 12 and 13. A nucleic acid encoding a polypeptide which is an amino acid sequence mutant, variant, derivative or allele of the sequence shown in SEQ ID NO: 2 is further provided by the present invention. Nucleic acid encoding such a polypeptide may show greater than 60% identity with the coding sequence shown in SEQ ID NO: 1, greater than about 70% identity, greater than about 80% identity, greater than about 90% identity or greater than about 95% identity. Nucleic acid encoding a variant or truncation of SEQ ID NO: 2, such as any one of those depicted in FIG. 12 are also encompassed herein and are designated with respect to SEQ ID NO: 2 as: A: 22-101 aa of FL; B: 1-21 aa linked to 38-101 aa; C: 1-37 aa linked to 49-101 aa; D: 1-48 aa linked to 58-101 aa; E: 1-57 aa linked to 72-101 aa; F: 1-77 aa linked to 81-101 aa; and G: 1-72 aa. The amino acid sequences of exemplary Tat variants are also shown in FIG. 13.

The present invention provides a method of obtaining a nucleic acid of interest, the method including hybridization of a probe having part or all of the sequence shown in SEQ ID NO: 1 or a complementary sequence, to target nucleic acid. Successful hybridization leads to isolation of nucleic acid which has hybridized to the probe, which may involve one or more steps of polymerase chain reaction (PCR) amplification.

Such oligonucleotide probes or primers, as well as the full-length sequence (and mutants, alleles, variants, and derivatives) are useful in screening a test sample containing nucleic acid for the presence of mutants or variants of a Tat polypeptide, the probes hybridizing with a target sequence from a sample obtained from a cell, tissue, or organism being tested. The conditions of the hybridization can be controlled to minimize non-specific binding. Preferably stringent to moderately stringent hybridization conditions are used. The skilled person is readily able to design such probes, label them and devise suitable conditions for hybridization reactions, assisted by textbooks such as Sambrook et al (1989) and Ausubel et al (1992).

In some preferred embodiments, oligonucleotides according to the present invention that are fragments of the sequences shown in SEQ ID NO: 1, are at least about 10 nucleotides in length, more preferably at least 15 nucleotides in length, more preferably at least about 20 nucleotides in length. Such fragments themselves individually represent aspects of the present invention. Fragments and other oligonucleotides may be used as primers or probes as discussed but may also be generated (e.g. by PCR) in methods concerned with determining the presence in a test sample of a sequence encoding a Tat variant.

### B. Proteins

A full-length Tat protein of the present invention may be prepared in a variety of ways, according to known methods. The protein may be purified from appropriate sources. This is not, however, a preferred method due to the low amount of protein likely to be present in a given cell type at any time. The availability of nucleic acid molecules encoding Tat enables production of the protein using in vitro expression methods known in the art. For example, a cDNA or gene may be cloned into an appropriate in vitro transcription vector, such as pSP64 or pSP65 for in vitro transcription, followed by cell-free translation in a suitable cell-free translation system, such as wheat germ or rabbit reticulocyte lysates. In vitro transcription and translation systems are commercially available, e.g., from Promega Biotech, Madison, Wis. or BRL, Rockville, Md.

Alternatively, according to a preferred embodiment, larger quantities of Tat may be produced by expression in a suitable prokaryotic or eukaryotic system. For example, part or all of a DNA molecule, such as SEQ ID NO: 1, may be inserted into a plasmid vector adapted for expression in a bacterial cell, such as E. coli. Such vectors comprise regulatory elements necessary for expression of the DNA in a host cell (e.g. E. coli) positioned in such a manner as to permit expression of the DNA in the host cell. Such regulatory elements required for expression include promoter sequences, transcription initiation sequences and, optionally, enhancer sequences.

The Tat polypeptide produced by gene expression in a recombinant prokaryotic or eukaryotic system may be purified according to methods known in the art. In a preferred embodiment, a commercially available expression/secretion system can be used, whereby the recombinant protein is expressed and thereafter secreted from the host cell, to be easily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Such methods are commonly used by skilled practitioners.

The Tat proteins of the invention, prepared by the aforementioned methods, may be analyzed according to standard procedures. For example, such proteins may be subjected to amino acid sequence analysis, according to known methods.

Polypeptides which are amino acid sequence variants, derivatives or mutants are also provided by the present invention. A polypeptide which is a variant, derivative, or mutant may have an amino acid sequence that differs from that given in SEQ ID NO: 2 by one or more of addition, substitution, deletion and insertion of one or more amino acids. Preferred such polypeptides have Tat function, that is to say have one or more of the following properties: the ability to induce CD154 release from platelets; the ability to promote enhanced immune responses to infections via platelet activation; the ability to induce platelet activation via interaction with CCR3; and immunological cross-reactivity with an antibody reactive with the polypeptide for which the sequence is given in SEQ ID NO: 2; and sharing an epitope with the polypeptide for which the sequence is given in SEQ ID NO: 2 (as determined for example by immunological cross-reactivity between the two polypeptides.

Modulation of platelet activity may also be effected using a CCR3 antagonist or agonist in accordance with the present invention. In accordance with the invention, a method is described wherein a CCR3 antagonist is used to inhibit platelet activation. Such methods may be performed in vitro or in vivo. CCR3 antagonists are known in the art and include, without limitation, SB-297006; SB-328437; GW701897B; YM-344031; DPC168 (a benzylpiperidine-substituted aryl urea CCR3 antagonist) and its acetylpiperidine derivative, BMS-570520; BMS-639623; and pyrazolone methylamino piperidine derivatives. See, for example, Erin et al. (2002, Curr Drug Targets Inflamm Allergy 1:201-214), Pruitt et al. (2007, Bioorganic & Medicinal Chemistry Letters, 17: 2992-2997), Santella III et al. (2008, Bioorganic & Medicinal Chemistry Letters, Volume 18:576-585), Pegurier et al. (2007, Bioorganic & Medicinal Chemistry Letters, Volume 17:4228-4231), and Wacker et al. (2002, Bioorganic & Medicinal Chemistry Letters, Volume 12:1785-1789), which are incorporated herein in their entireties. In a further embodiment, a method is described wherein a CCR3 agonist is used to promote platelet activation. CCR3 agonists are known in the art and include, without limitation, TY-18526 and CH0076989. See, for example, Masayuki et al. (2004, Allergy in Practice 322:792-795), Wise et al. (2007, J Biol Chem 282:27935-27943), and Ting et al. (2005, Bioorganic and Medicinal Chem Letters 15:3020-3023), which are incorporated herein in their entireties.

A polypeptide which is an amino acid sequence variant, derivative or mutant of the amino acid sequence shown in SEQ ID NO: 2 may comprise an amino acid sequence which shares greater than about 35% sequence identity with the sequence shown, greater than about 40%, greater than about 50%, greater than about 60%, greater than about 70%, greater than about 80%, greater than about 90% or greater than about 95%. Particular amino acid sequence variants may differ from that shown in SEQ ID NO: 2 by insertion, addition, substitution or deletion of 1 amino acid, 2, 3, 4, 5-10, 10-20, 20-30, 30-40, 40-50, 50-100, 100-150, or more than 150 amino acids. For amino acid “homology”, this may be understood to be identity or similarity (according to the established principles of amino acid similarity, e.g., as determined using the algorithm GAP (Genetics Computer Group, Madison, Wis.). GAP uses the Needleman and Wunsch algorithm to align two complete sequences that maximizes the number of matches and minimizes the number of gaps. Generally, the default parameters are used, with a gap creation penalty=12 and gap extension penalty=4. Use of GAP may be preferred but other algorithms may be used including without limitation, BLAST (Altschul et al. (1990 J. Mol. Biol. 215:405-410); FASTA (Pearson and Lipman (1998) PNAS USA 85:2444-2448) or the Smith Waterman alogrithm (Smith and Waterman (1981) J. Mol. Biol. 147:195-197) generally employing default parameters. Use of either of the terms “homology” and “homologous” herein does not imply any necessary evolutionary relationship between the compared sequences. The terms are used similarly to the phrase “homologous recombination”, i.e., the terms merely require that the two nucleotide sequences are sufficiently similar to recombine under appropriate conditions.

A polypeptide according to the present invention may be used in screening for molecules which affect or modulate its activity or function. Such molecules may be useful for research purposes.

### II. Uses of Tat Polypeptide-Encoding Nucleic Acids and Tat Polypeptides

Tat polypeptide is a regulatory protein of HIV, which may be used to advantage to enhance immune responses to viral or bacterial infections in an organism or subject. Moreover, expression of Tat may be specifically targeted to a particular tissue or tissues in a subject so as to specifically enhance immune responses to viral or bacterial infections in the targeted tissue(s). Accordingly, Tat molecules and compositions of the invention may be used to advantage to treat a patient with a viral or bacterial infection. Such viral infections include hepatitis virus (e.g., hepatitis A, hepatitis B, hepatitis C, hepatitis D, and hepatitis E viruses), herpesviruses (e.g. herpes simplex virus types 1 and 2, varicella-zoster virus, cytomegalovirus, Epstein Barr virus, and human herpes viruses types 6, 7, and 8), and influenza virus. Such bacterial infections include, without limitation, Gram-positive cocci, for example Staphylococci (Staph. aureus, Staph. epidermidis) and Streptococci (Strept. agalactiae, Strept. faecalis, Strept. pneumoniae, Strept. pyogenes); Gram-negative cocci (Neisseria gonorrhoeae and Yersinia pestis) and Gram-negative rods such as Enterobacteriaceae, for example Escherichia coli, Hamophilus influenzae, Citrobacter (Citrob. freundii, Citrob. divernis), Salmonella and Shigella, and Francisella (Francisella tularensis); Gram-positive rods such as Bacillus (Bacillus anthracis, Bacillus thuringenesis); furthermore Klebsiella (Klebs. pneumoniae, Klebs. oxytoca), Enterobacter (Ent. aerogenes, Ent. agglomerans), Hafnia, Serratia (Serr. marcescens), Proteus (Pr. mirabilis, Pr. rettgeri, Pr. vulgaris), Providencia, Yersinia, and the genus Acinetobacter. Also encompassed are bacterial infections caused by the genus Pseudomonas (Ps. aeruginosa, Ps. maltophilia) and strictly anaerobic bacteria such as, for example, Bacteroides fragilis, representatives of the genus Peptococcus, Peptostreptococcus and the genus Clostridium; furthermore Mycoplasmas (M. pneumoniae, M. hominis, Ureaplasma urealyticum) as well as Mycobacteria, for example Mycobacterium tuberculosis.

### A. Tat-Encoding Nucleic Acids

Tat-encoding nucleic acids may be used for a variety of purposes in accordance with the present invention. Tat-encoding DNA, RNA, or fragments thereof may be used as probes to detect the presence of and/or expression of genes encoding Tat-like proteins. Methods in which Tat-encoding nucleic acids may be utilized as probes for such assays include, but are not limited to: (1) in situ hybridization; (2) Southern hybridization (3) northern hybridization; and (4) assorted amplification reactions such as PCR.

The Tat-encoding nucleic acids of the invention may also be utilized as probes to identify related genes from other viral strains. As is well known in the art, hybridization stringencies may be adjusted to allow hybridization of nucleic acid probes with complementary sequences of varying degrees of homology. Thus, Tat-encoding nucleic acids may be used to advantage to identify and characterize other genes of varying degrees of relation to Tat, thereby enabling further characterization of the immune modulatory capabilities of Tat. Additionally, they may be used to identify genes encoding proteins that interact with tat (e.g., by the “interaction trap” technique), which should further accelerate identification of the components involved in immune regulation/modulation.

Nucleic acid molecules, or fragments thereof, encoding Tat may also be utilized to control the production of Tat, thereby regulating the amount of protein available to participate in immune regulation. Alterations in the physiological amount of Tat protein may dramatically affect the activity of other protein factors involved in immune regulation.

### B. Tat Protein

Purified Tat protein, or a variant, derivative, or fragment thereof, produced via expression of Tat encoding nucleic acids of the present invention may be used to advantage to enhance immune responses to viral and/or bacterial infection in a cell, tissue, or organism, as discussed above.

From the foregoing discussion, it can be seen that Tat-encoding nucleic acids and Tat expressing vectors can be used to produce large quantities of Tat protein and alter Tat protein accumulation for the purposes of therapeutic intervention in the treatment of viral and/or bacterial infections.

The present inventors have made the surprising discovery that Tat polypeptides can be administered to a patient to enhance immune responses to microbial infections, particularly those caused by bacteria or viruses that do not express Tat polypeptide. As described herein, Tat has been shown to be an enhancer of platelet activation. Of note, prior to the discovery of the present invention, the potential for using Tat as an enhancer of immune responses to bacterial infections or non-Tat expressing viral infections had not be appreciated. As shown herein, Tat enhances immune responses to microbial infections by interacting with platelet CCR3 and thereby activating platelets. Such activity may effectuate a partial or total reduction in symptoms associated with a microbial infection.

The novel findings of the present inventors, therefore, present new applications for which Tat nucleic and amino acid sequences and compositions thereof may be used to advantage. Such utilities include, but are not limited to, various therapeutic applications as described herein. Also described is a kit comprising Tat nucleic and/or amino acid sequences, Tat activity compatible buffers, and instruction materials.

Tat polypeptides for use in the present methods comprise the amino acid sequence of SEQ ID NO: 2 [Full length (FL) Tat is 101 amino acids (aa); see FIG. 12]. Additional variants and truncations of SEQ ID NO: 2, as depicted in FIG. 12, are designated with respect to SEQ ID NO: 2 as: A: 22-101 aa of FL; B: 1-21 aa linked to 38-101 aa; C: 1-37 aa linked to 49-101 aa; D: 1-48 aa linked to 58-101 aa; E: 1-57 aa linked to 72-101 aa; F: 1-77 aa linked to 81-101 aa; and G: 1-72 aa and are also envisioned. The amino acid sequences of additional Tat variants are presented in FIG. 13.

### HIV-1 Tat Variants and Platelet Activation

The potential role of platelet activation in HIV-1 infection has been investigated using platelet samples from 15 controls and 20 HIV-1-infected patients with normal platelet counts. Platelet activation was examined by using flow cytometry to assess platelet activation markers (P-selectin, CD63) and the amount of platelet derived microvesicles. Enhanced activation of platelets was found in HIV-1-infected patients group and was strongly correlated with the plasma viral load (Holme et al. FASEB J. 1998; 12:79-89). The mechanism of how HIV infection induces platelet activation, however, remained unknown.

The chemokine activity of Tat was first identified by noting that Tat is a strong chemoattractant for monocytes (Albini et al. J Biol Chem. 1998; 273:15895-15900). This chemotaxis of monocytes towards Tat was blocked by pertussis toxin but not cholera toxin, indicating involvement of Gi proteins. Peptide mapping of the entire Tat protein showed that the monocyte chemotactic activity was concentrated in the cysteine-rich and core domains of Tat (Noonan et al. Adv Pharmacol. 2000; 48:229-250). These domains are the most highly conserved domains of the Tat protein and contain both CC and CXC motifs, which show some sequence similarity with other chemokines. Consequently, it was shown that Tat interacted with β-chemokine receptors CCR2 and CCR3, but not with CCR1, CCR4, and CCR5 (Albini et al. J Biol Chem. 1998; 273:15895-15900; Albini et al. Proc Natl Acad Sci USA. 1998; 95:13153-13158; Mitola et al. Blood. 1997; 90:1365-1372). CCR3 is expressed on eosinophils, mast cells, basophils, a subset of TH2 T cells and platelets (Clemetson et al. Blood. 2000; 96:4046-4054; Forsythe et al. Am J Respir Cell Mol Biol. 2003; 28:405-409). It has been suggested that Tat interacts with mast cells through CCR3, and may contribute to high IgE level in HIV-1-infected patients (Marone et al. Trends Immunol. 2001; 22:229-232). Although platelets express CCR3, prior to the present invention it was not clear that Tat was able to interact with the platelet through CCR3 and, moreover, the ramifications of such a potential interaction were not known. The role of β3 integrin in Tat induced platelet activation was also unappreciated prior to the present invention. In light of the above, the combined contribution of CCR3 and β3 integrin in Tat induced platelet activation was also unrealized prior to the instant invention.

Tat variants are found in different HIV strains (Noonan et al. Adv Pharmacol. 2000; 48:229-250; Pugliese et al. Cell Biochem Funct. 2005; 23:223-227; Pantano et al. Proteins. 2005; 58:638-643). Although both chemokine binding motif (CCF) and integrin binding motif (RGD) in Tat are relatively conserved, it is not clear whether there are differences among Tat variants with respect to their ability to induce platelet activation. More particularly, unanswered questions remain as to which domain(s) of Tat is(are) required for Tat induced platelet activation and if different Tat variants induce different types or degrees of platelet activation. In fact, the variation of platelet activation among HIV-1 infected patient and the variation of severity of autoimmune response in different HIV-1 patient strongly suggest that there are differences among Tat variants in inducing platelet activation. Thus, the potential usage of a specific Tat variant to induce platelet activation has not been appreciated before.

Based on results presented herein, the CCR3 binding motif and the RGD motif, which participates in integrin binding, are thought to be important for Tat induced platelet activation.

### C. Therapeutic Uses of Tat Polypeptides

The invention provides for treatment of infectious diseases by administration of a therapeutic compound identified through the above described methods. Such compounds include but are not limited to proteins, peptides, protein or peptide derivatives or analogs, antibodies, nucleic acids, and small molecules.

The invention provides methods for treating patients afflicted with an infectious disease comprising administering to a subject an effective amount of a compound identified by the method of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), and construction of a nucleic acid as part of a retroviral or other vector. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally, e.g., by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into a localized site of a bacterial infection, such as, for example, a boil or abcess.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, J., 1983, Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., a target tissue or tumor, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

### D. Therapeutic Uses of Nucleic Acid Sequences Encoding Tat Polypeptides

Methods for administering and expressing a nucleic acid sequence are generally known in the area of gene therapy. For general reviews of the methods of gene therapy, see Goldspiel et al. (1993) Clinical Pharmacy 12:488-505; Wu and Wu (1991) Biotherapy 3:87-95; Tolstoshev (1993) Ann. Rev. Pharmacol. Toxicol. 32:573-596; Mulligan (1993) Science 260:926-932; and Morgan and Anderson (1993) Ann. Rev. Biochem. 62:191-217; May (1993) TIBTECH 11(5): 155-215. Methods commonly known in the art of recombinant DNA technology which can be used in the present invention are described in Ausubel et al. (eds.), 1993, Current Protocols in Molecular Biology, John Wiley & Sons, NY; and Kriegler (1990) Gene Transfer and Expression, A Laboratory Manual, Stockton Press, NY.

In a particular aspect, a nucleic acid encoding a Tat polypeptide or variant, derivative, or fragment thereof is incorporated into an expression vector that expresses the Tat polypeptide or variant, derivative, or fragment thereof in a suitable host. In particular, such a nucleic acid has a promoter operably linked to the coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller and Smithies (1989) Proc. Natl. Acad. Sci. USA 86:8932-8935; Zijlstra et al. (1989) Nature 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject, known as “ex vivo gene therapy”.

In another embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, 1987, J. Biol. Chem. 262:4429-4432), which can be used to target cell types specifically expressing the receptors.

In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al. (1989) Nature 342:435-438).

In a further embodiment, a retroviral vector can be used (see Miller et al. (1993) Meth. Enzymol. 217:581-599). These retroviral vectors have been modified to delete retroviral sequences that are not necessary for packaging of the viral genome and integration into host cell DNA. More detail about retroviral vectors can be found in Boesen et al. (1994) Biotherapy 6:291-302, which describes the use of a retroviral vector to deliver the mdr1 gene to hematopoietic stem cells in order to make the stem cells more resistant to chemotherapy. Other references illustrating the use of retroviral vectors in gene therapy are:, Clowes et al. (1994) J. Clin. Invest. 93:644-651; Kiem et al. (1994) Blood 83:1467-1473; Salmons and Gunzberg (1993) Human Gene Therapy 4:129-141; and Grossman and Wilson (1993) Curr. Opin. in Genetics and Devel. 3:110-114.

Adenoviruses may also be used effectively in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (1993) Current Opinion in Genetics and Development 3:499-503 present a review of adenovirus-based gene therapy. Bout et al. (1994) Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al. (1991) Science 252:431-434; Rosenfeld et al. (1992) Cell 68:143-155; Mastrangeli et al. (1993) J. Clin. Invest. 91:225-234; PCT Publication WO94/12649; and Wang, et al. (1995) Gene Therapy 2:775-783. Adeno-associated virus (AAV) has also been proposed for use in gene therapy (Walsh et al. (1993) Proc. Soc. Exp. Biol. Med. 204:289-300; U.S. Pat. No. 5,436,146).

Another suitable approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr (1993) Meth. Enzymol. 217:599-618; Cohen et al. (1993) Meth. Enzymol. 217:618-644; Cline (1985) Pharmac. Ther. 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject; recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver. In a particular embodiment, the cell used for gene therapy is autologous to the subject that is treated.

In another embodiment, the nucleic acid to be introduced for purposes of gene therapy may comprise an inducible promoter operably linked to the coding region, such that expression of the nucleic acid is controllable by adjusting the concentration of an appropriate inducer of transcription.

Direct injection of a nucleic acid sequence encoding a Tat polypeptide may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection.

### E. Pharmaceutical Compositions

The present invention also provides pharmaceutical compositions. Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin, incorporated in its entirety by reference herein. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of an infectious disease (e.g., a disease caused by a virus or bacteria) can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

### F. Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

The following protocols are provided to facilitate the practice of the present invention.

The following examples are put forth so as to provide those of ordinary skill in the art with a complete disclosure and description of how to make and use the assay, screening, and therapeutic methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### EXAMPLE I

### Materials and Methods

### Materials

All reagents were obtained from Sigma (St. Louis, Mo.) unless otherwise designated. HPLC purified HIV-1 Tat protein is from Immunodiagnostics, Inc (Scottsdale, Ariz.). CD154−/−mice, β3−/−mice with C57BL/6J background, C57BL/6J and BALB/c wild type mice were obtained from Jackson Laboratory. The genotype of knockout mice was confirmed by PCR. The animal protocol was approved by the New York University Medical Center Institutional Review Board. The following reagents were obtained through the NIH AIDS Research and Reference Reagent Program, Division of AIDS, NIAID, NIH: pcDNA3.1+/tat101-flag from Dr. Eric Verdin and pGEX2T-Tat from Dr. Andrew Rice. Control plasmids pGEX4T-2 were obtained from GE Healthcare (Piscataway, N.J. 08855) and pET28b-Adamts18-66 was previously constructed in the lab (Li et al. 2009, Blood 113: 6051-60). CCR3 inhibitor SB328437 is from Calbiochem (San Diego, Calif.). CCL5 (Recombinant Mouse Rantes) is from Shenandoah Biotechnology, Inc (Warwick, Pa.). The adenovirus vector was kindly provided by Dr.Chuanju Liu from Hospital for Joint Diseases of New York University Medical Center. Retrovirus vector MSCV-puro is from Clontech (Mountain View, Calif.).

### Tat Cloning, Expression and Purification

### Tat Cloning

Two clone vectors have been used to express TAT in both E. coli and eukaryotic cells, respectively. The Tat gene (86aa) contained in a pGEX2T-Tat plasmid was subcloned into the expression vector pET28b and the Tat gene (101aa) from pcDNA3.1+/tat101-flag plasmid was subcloned into retroviral vector MSCV-puro.

### Tat Expression and Purification

Rosetta BL21 (DE3) competent cells were transformed with pGEX2T-Tat and pET28b-Tat. Bacterial clones were cultured in 2YT medium until A600 reached 0.3-0.6. The expression of GST-Tat was induced by IPTG (Isopropyl-β-D-thio-galactoside). GST-Tat was purified using GST purification modules (GE Healthcare). Trace contamination of endotoxin was removed by Endotoxin Removing Gel (Thermo Scientific, Rockford, Ill.). Control proteins GST from pGEX4T-2 and His-Ad18-66 from pET28b-Adamts18-66 were prepared parallel with Tat protein to ensure no artifact due to contamination.

### Flow Cytometry Assay of Platelet Activation

Gel-filtered human or mouse platelets were prepared from platelet-rich plasma obtained from blood collected in 0.38% sodium citrate via heart puncture. 1×10⁷gel-filtered platelets were incubated under different stimulation conditions at 37° C. for 30 minutes. For in vitro platelet activation, Tat 20 ng/ml, CCL5 10 μg/ml, or control Ad18-66 20 ng/ml were used. To test the role of chemokine receptor CCR3, calcium flux and cAMP in Tat-induced platelet activation, CCR3 inhibitor SB328437 3 nM, calcium flux inhibitor EGTA 100 μM and prostaglandin E1 (PGE1, a cAMP elevator) 5 μM were used separately to treat platelets at 37° C. for 30 minutes before incubation with Tat, CCL5 or control as above. To test the role of β3 integrin, platelets from β3 integrin knock out mice were incubated with Tat, CCL5, thrombin, and control. Treated platelets were then stained with anti-CD154-FITC and anti-CD62P-PE antibody on ice for one hour. Fluorescent-labeled platelets were measured by flow cytometry (FACScan, Becton-Dickinson, Calif.).

### In Vivo Tat Expression Stimulate Platelet Activation

Transfection of 4T1 Cells with MSCV-Tat and Control MSCV-TatR

MSCV-Tat and MSCV-TatR (reversed insertion Tat as control) pseudo-retrovirus were generated and concentrated by ultracentrifugation as described (Hawley et al. Gene Ther. 1994; 1: 136-8; Soneoka et al. Nucleic Acids Res. 1995; 23: 628-33). 4T1 cells (a carcinoma cell line syngeneic with BALB/c mouse) were infected with MSCV-Tat or MSCV-TatR pseudo-retrovirus in 35 mm dishes. Cells were selected with puromycin (3 μg/ml, InvivoGen). Positive 4T1 cells were collected to confirm the expression of Tat by both RT-PCR and Western Blot.

### Injection 4T1 Cells and Pseudo-Retrovirus to BALB/c Mice

5×10⁶ positive 4T1 cells (transfected with MSCV-Tat or MSCV-TatR) were injected into BALB/c mice by intraperitoneal injection. 24 hours after the injection, and platelets were collected in Tyrode's buffer for flow cytometry assay as above.

Concentrated MSCV-Tat or MSCV(control) pseudo-retrovirus particles were injected into mice via tail vein to mimic HIV infection. Platelets were collected for flow cytometry assay two weeks after three times injection. Serum level of Tat was examined by Western Blot.

Sera were collected from both 4T1 cell line and pseudo-retrovirus injected mice. The titer of soluble IgG1 and IgG2b were detected with anti-mouse IgG1-HRP and anti-mouse IgG2b-HRP mAb (Santa Cruz Biotech, Inc, Santa Cruz, Calif.) by ELISA.

Tat Interaction with Platelets

### S³⁵ Labeling of Tat

S³⁵-methionine labeled Tat and control protein were translated from pET28b/Tat and luciferase T7 control DNA plasmids using an in vitro translation kit (TNT® Coupled Reticulocyte Lysate Systems, Promega, Madison, Wis. 53711) following the manufacturer's protocol. The S³⁵-Tat or S³⁵-luciferase proteins were incubated with 1×10⁷ platelets for 30 minutes at room temperature. Platelets were then washed with PBS three times to remove unbound protein. The total protein of the platelets was extracted and resolved on SDS-PAGE gel. The film was exposed to the dried gel at 4° C. for two weeks to detect binding of Tat to platelets.

### GST-Tat Pulling Down of β3 Integrin

Tat interaction with the β3 integrin was determined by incubating purified GST-Tat protein or GST protein (5 μg) with the protein lysates from 1×10⁸ platelets 2 hours at 4° C. Glutathione-conjugated beads were used to pull down the GST-Tat or GST. The total proteins were resolved on the SDS-PAGE gel and transferred to PVDF membranes for western blotting to detect β3 integrin protein.

### Measure CD154 Release in Tat-Treated Platelets

To assess Tat-induced platelet CD154 release, fresh isolated mouse platelets were incubated with Tat or control Ad18-66. Supernatant were collected and concentrated for Western blot to detect CD154.

### Electron Microscopy

1×10⁷ gel-filtered mouse platelets platelets were incubated with Tat 20 ng/ml or Ad18-66 20 ng/ml at 37° C. for 30 minutes, followed by fixation in an equal volume of 0.1% glutaraldehyde in White saline (White. Methods Mol Biol. 2004; 272: 47-630). After fixation and dehydration, the platelet sample was embedded in an Epon/Aarldite resin mixture. Sample sections were cut using an ultramicrotome and mounted on copper grids for EM images in the Imagine Core Facility of Skirball Institute of Biomolecular Medicine at NYU.

### In Vitro Induction of B Cell Activity

Splenic B cells were isolated from RBC-depleted splenocytes of CD154^{-/-} mice and cultured at 1×10⁶ cell/ml in 10% FCS-RPMI 1640 with 0.05 mM 2-ME, plus IL-4 (100 U/ml) and IL-10 (100 ng/ml). 1×10⁸ platelets treated with Tat 20 ng/ml or control Ad18-66 20 ng/ml were then added into the splenic B cell culture. Cells were collected on day 5 and double stained with anti-mouse IgG1-FITC mAb or anti-mouse IgG2b-FITC mAb and anti-mouse CD45R-PE mAb (BD Biosciences, San Jose, Calif.) for analysis of surface Ig expression on a FACSCalibur flow cytometry (BD Biosciences, San Jose, Calif.) (Cerutti et al. J Immunol. 1998; 160: 2145-57).

### Antibody Generation and B Cell Activity in Vivo

Immunization of Balb/c Mice with Adenovirus

5×10⁶ 293-HEK cells were infected with adenoviruses (AdEasy adenoviral vector system, Stratagene, Santa Clara, Calif.). Adenovirus infected cells were frozen and thawed three times to collect supernatant for virus. Viruses were inactivated by heating to 100° C. for 10 minutes before mixing with an equal volume of mineral oil for immunization. BALB/c mice were injected with Tat (50 ng) or control Ad18-66 (50 ng) in 100 PBS through the tail before immunization with 100 ul of antigen mixture (about 1×10⁸ inactive viruses) by intraperitoneal injection. Tat and control Ad18-66 were injected every two days and the immunization was boosted every four days until day 12. Serum from each mouse was collected from mouse tail every two days to monitor the anti-adenovirus antibody titer by ELISA.

Flow Cytometry of B Cell from BALB/c Mice

To examine B cell activity in vivo, BALB/c mice were immunized and treated with Tat and control Ad18-66 as above. B cells were collected from the spleen at day 6. Purified B cells were double stained with anti-mouse IgG1-FITC or anti-mouse IgG2b-FITC mAb and anti-mouse CD45R-PE mAb and analyzed with a FACSCalibur flow cytometry.

### Statistical Analysis

All results were expressed as mean value plus or minus standard deviation (SD). The statistical significance was determined using student's T-test. The number of mice or samples included in each statistical analysis is specified in the figure legends.

### Results

### Tat Activates Platelets

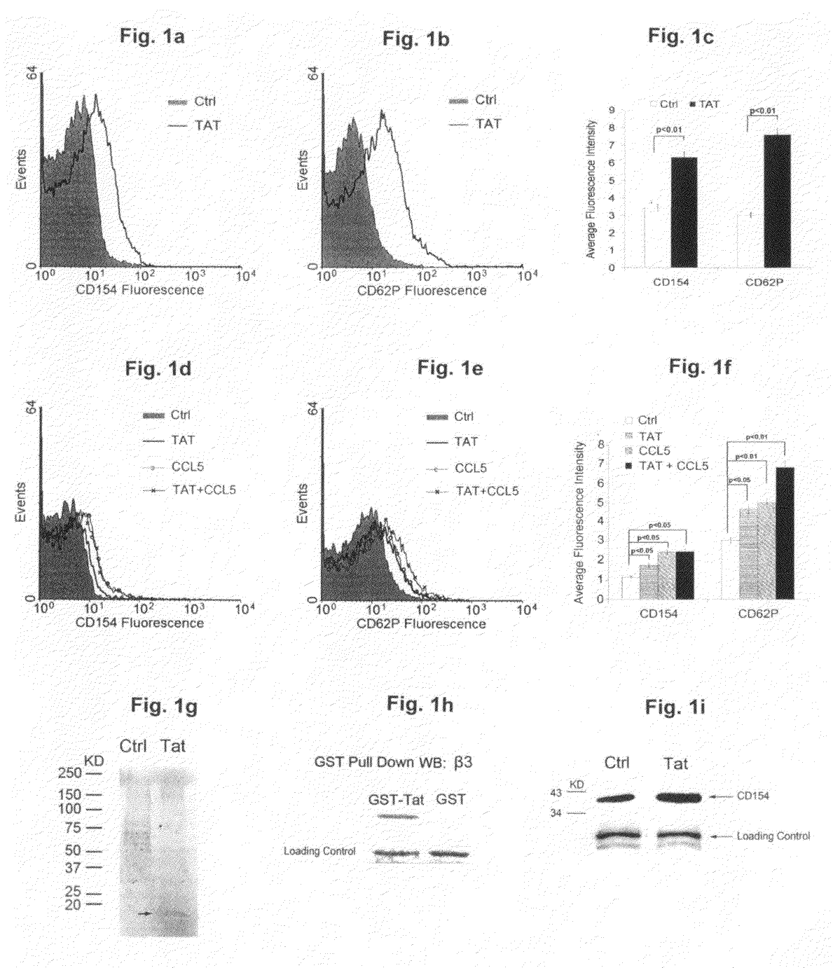

Tat has been reported to mimic the ligand of chemokine receptor CCR3 and to activate monocytes. Since platelets also express CCR3, the present inventor tested whether Tat is able to interact with CCR3 on the platelet surface and activate platelets. The concentration of Tat found in the serum of many HIV-1 positive patients is 2 ng to 40 ng/ml (Xiao et al. Proc Natl Acad Sci USA. 2000; 97: 11466-71). Platelets were, therefore, treated with 20 ng/ml and platelet activation was measured by assessing the expression of the well-described platelet activation markers CD62P and CD154 by flow cytometry. As shown in FIG. 1(a,b,c), Tat is able to induce platelet activation as compared to the control protein AD18-66. To confirm that CCR3 is involved in Tat-induced platelet activation, Tat-induced platelet activation was compared with platelet activation induced by the CCR3 ligand chemokine CCL5. Tat and CCL5 induce similar platelet activation while additional CD62P expression was found in the Tat plus CCL5 group (FIG. 1d,e,f).

Tat Interacts with Platelet Integrin β3 to Release CD154

Tat has previously been reported to interact with integrin αVβ3 by Tat's RGD motif (Urbinati et al. J Cell Sci. 2005; 118: 3949-58; Urbinati et al. Arterioscler Thromb Vasc Biol. 2005; 25: 2315-20). To test whether Tat is able to interact with platelet integrin β3, S³⁵-methionine labeled Tat or Luciferase proteins were incubated with human platelets. Unbound proteins were removed by washing with PBS. Platelets were then lysed to obtain total proteins and resolved by SDS-PAGE for autoradiography. As shown in FIG. 1g, a 14 KDa band Tat band was resolved in the gel, whereas luciferase was not, demonstrating that Tat specifically interacts with platelets. To further confirm that Tat is able to interact with integrin β3, GST-Tat was expressed in E. coli and purified with glutathione-conjugated beads. Total protein was extracted from platelets and was incubated with GST-Tat or GST protein followed by protein immunoprecipitation with glutathione-conjugated beads. The protein samples were subjected to Western Blot analysis to detect integrin β3 (FIG. 1h). A strong integrin β3 band was found in the GST-Tat pull down proteins, but not in the GST protein (control) pull down sample, suggesting that Tat can interact with integrin β3 on platelets. In addition, elevated CD154 levels were found in the supernatant of platelets incubated with Tat, suggesting that Tat not only induces the expression of CD154 on the surface of platelets, but also promotes the release of soluble CD154 (FIG. 1i).

### Tat-Induced Platelet Activation Requires Integrin β3 and CCR3

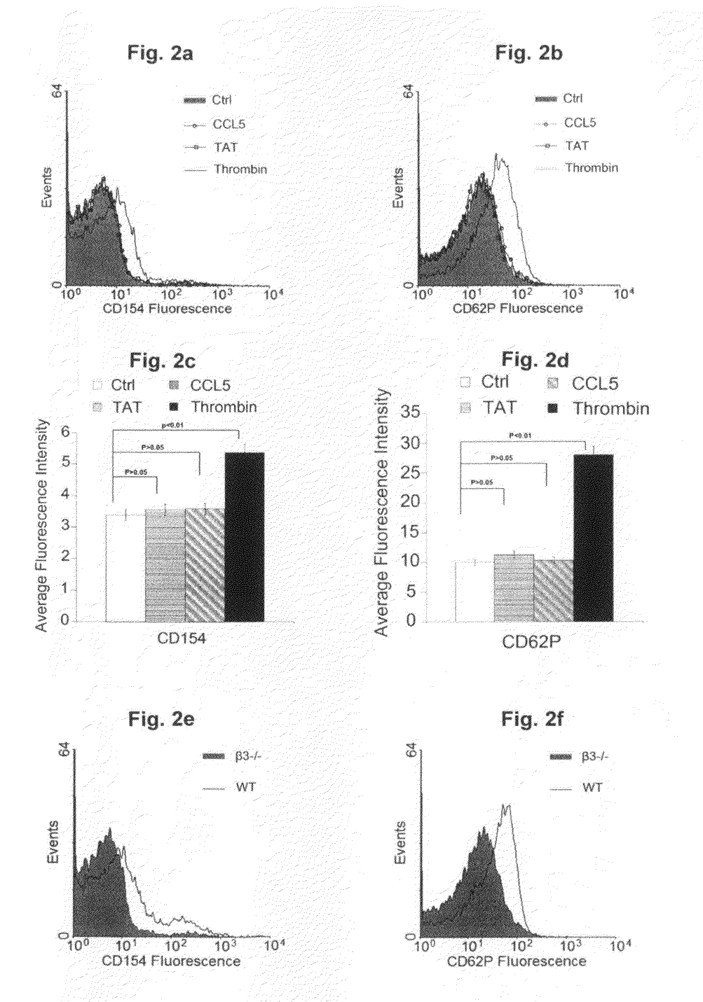

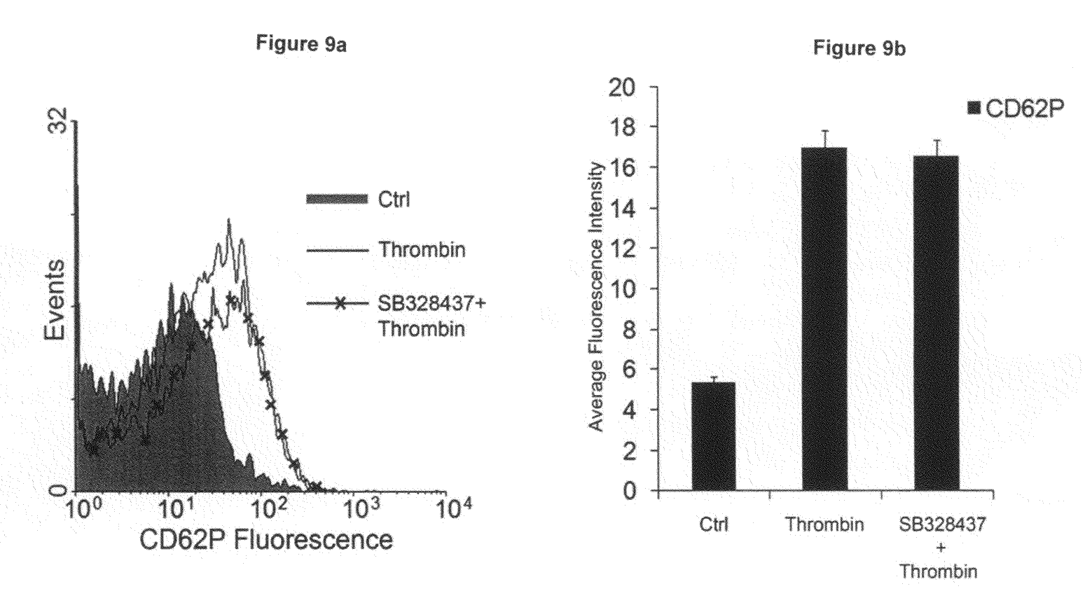

Since Tat is able to interact with both β3 integrin and CCR3 on platelets, the role of integrin β3 and CCR3 in Tat-induced platelet activation were next examined. Gel-filtered platelets from integrin β3 knockout mice (β3^{-/-}) were treated with Tat, CCL5 and thrombin. Flow cytometry analysis revealed lack of both CCL5 and Tat-induced platelet activation in integrin β3 deficient mouse platelets. In contrast, thrombin still induces platelet activation suggesting that Tat and CCL5 induced platelet activation requires integrin β3 but thrombin does not (FIGS. 2 and 9).

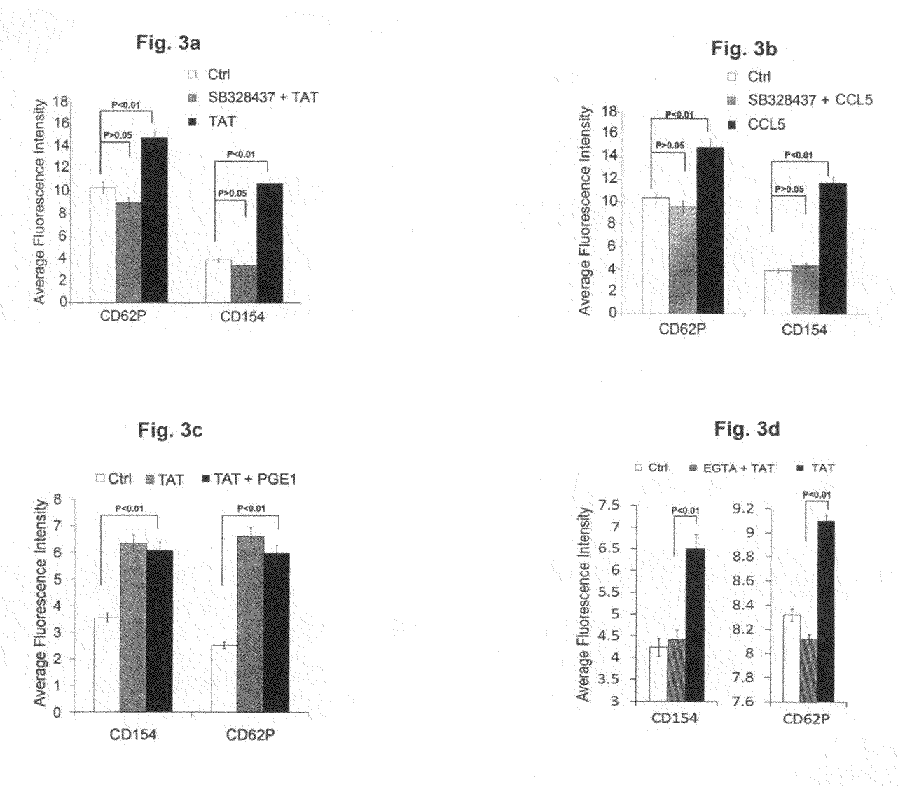

To examine the role of CCR3 in Tat-induced platelet activation, platelets were incubated with the CCR3 inhibitor SB328437 prior to exposure to Tat. SB328437 completely inhibited both Tat and CCL5 induced platelet activation but did not inhibit thrombin-induced platelet activation, suggesting that CCR3 is required for Tat-induced platelet activation but not required for thrombin-induced platelet activation (FIG. 3a,b; and FIG. 9).

Tat Induced Platelet Activation is Calcium Flux Dependent but cAMP Independent

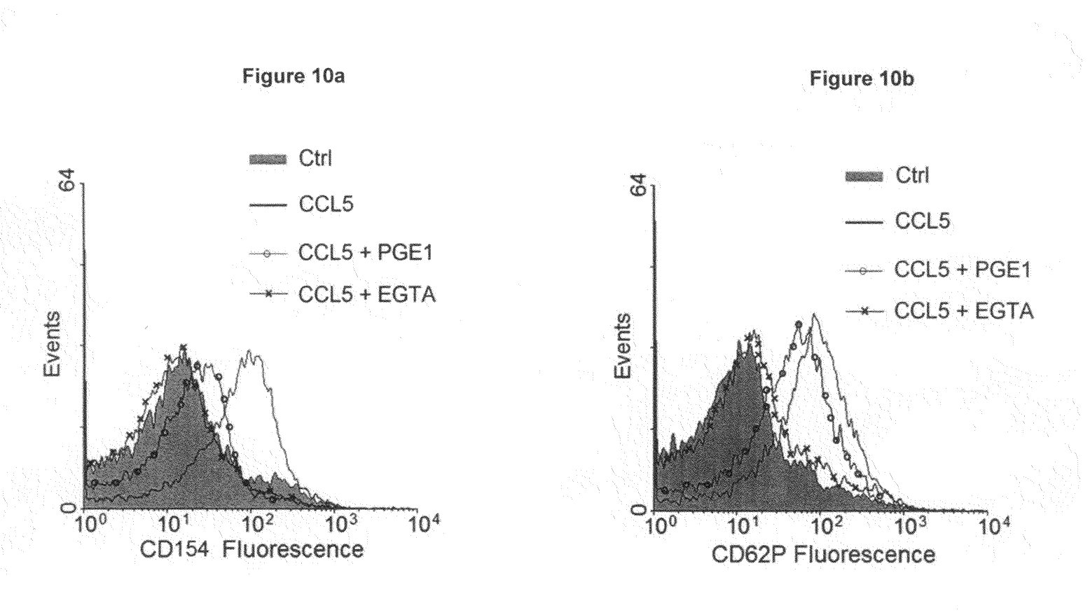

Since both calcium flux and cAMP are implicated in the regulation of platelet activation by a variety of agonists, the present inventor next examined their roles in Tat-induced platelet activation. Tat-treated platelets were incubated with and without prostaglandin E1 (PGE1) 5 μM, a cAMP elevator, and analyzed by flow cytometry (FIG. 3c). No significant inhibition was found with PGE1, suggesting that the effect of cAMP on Tat-induced platelet activation is not significant while PGE1 partially inhibited CCL5-induced platelet activation (FIG. 10). However, the calcium chelator EGTA completely inhibited both Tat and CCL5 induced platelet activation suggesting the role of calcium flux in Tat-induced platelet activation and a minor difference between Tat-induced platelet activation and CCL5-induced platelet activation (FIG. 3d, and FIG. 10).

### Tat Expression Cell Line and MSCV-Tat Virus Induce Platelet Activation in Vivo

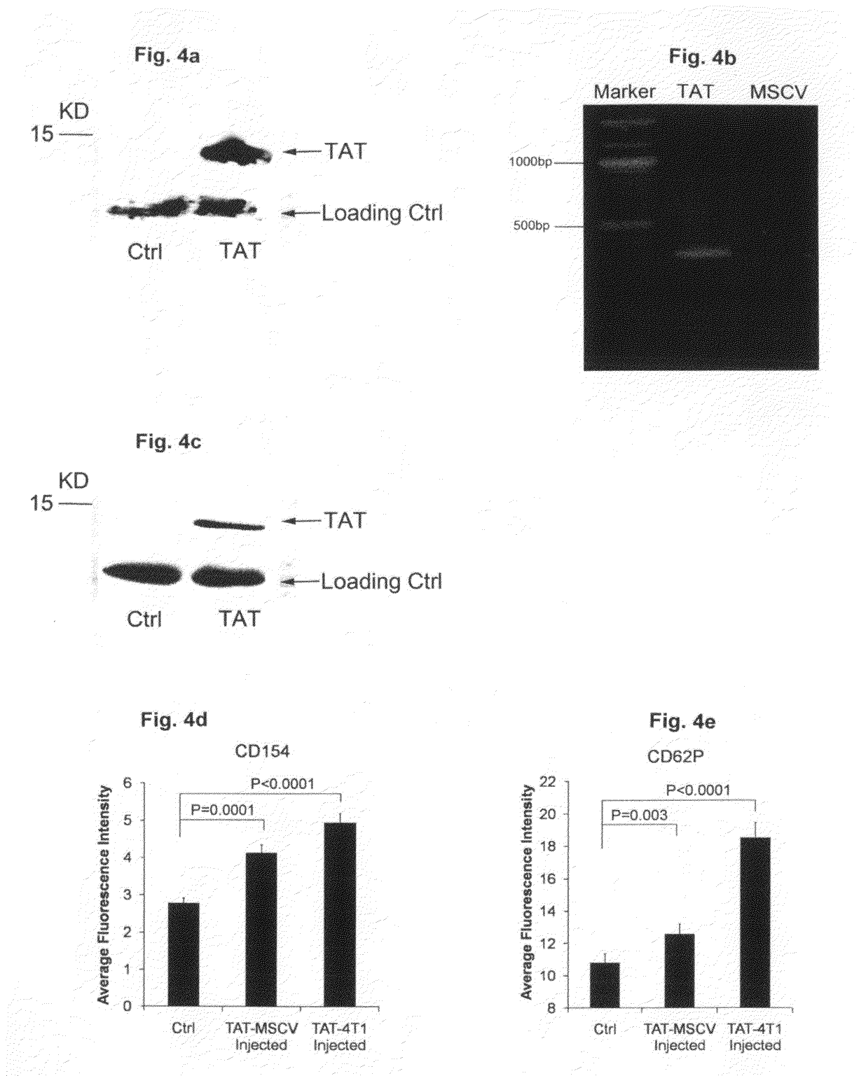

Although results presented herein demonstrate that the Tat protein is able to induce platelet activation and release CD154, it is not clear whether platelet activation observed in HIV-1 infected patients is indeed due to Tat expression. To determine whether Tat is able to induce platelet activation in vivo, the effect of Tat expressing cells and a Tat retrovirus on platelet activation in BALB/c mice was investigated. 4T1 cells (a carcinoma cell line syngeneic with BALB/c mouse) were transfected with full length Tat expression retrovirus (MSCV-Tat) or reverse insert Tat (MSCV-TatR) as a negative control. Tat expressing 4T1 cells or control 4T1 cells were injected intraperitoneally into BALB/c mice. Gel-filtered platelets were prepared at 24 hours, and platelet activation was assessed by flow cytometry. To mimic HIV-1 infection, MSCV-Tat retrovirus or MSCV retrovirus as control were also directly injected into BALB/c mice via tail vein every three days. Platelet activation was assessed by flow cytometry two weeks after the first injection of virus. The expression of Tat in 4T1 cells was confirmed by Western Blot and RT-PCR (FIG. 4a,b). Tat protein was elevated in the serum of MSCV-Tat infected BALB/c mice (FIG. 4c). Both 4T1-Tat cells and MSCV-Tat retrovirus induced platelet activation in vivo as demonstrated by (FIG. 4d,e), thus suggesting that Tat released by HIV-1 infected cells is able to induce platelet activation in vivo.

### Tat-Activated Platelets are Able to Induce B Activation Through Platelet Associated CD154 Both in Vitro and in Vivo

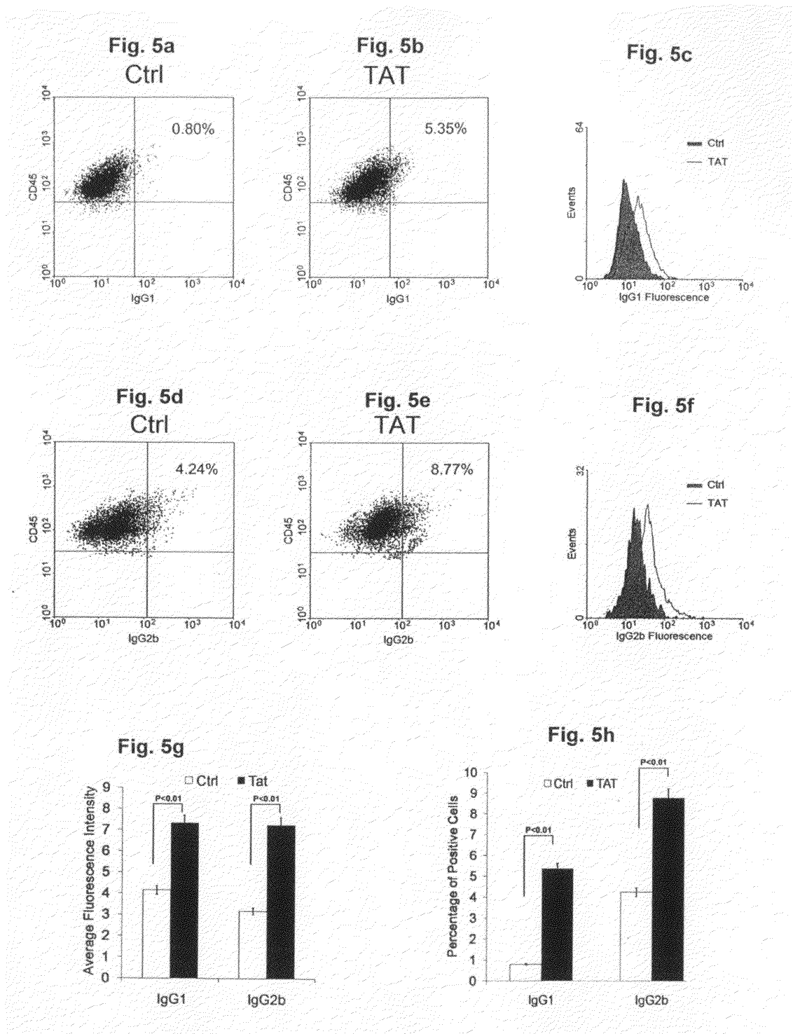

Since elevated CD154 correlates with the development of ITP (Meabed et al. Hematology. 2007; 12: 301-7; Solanilla et al. Blood. 2005; 105: 215-8) and activated platelets are known to release CD154, the present inventor next examined whether Tat-induced platelet-associated CD154 contributes to the development of autoantibody by promoting immunoglobulin production in B cells. To avoid endogenous CD154 from T cells, CD154⁴^{-/-}mouse spleen cells were co-cultured with wild type platelets (CD154^{+/+}) pretreated with Tat. Parallel co-cultures were setup with an Ad18-66 control protein. The surface IgG1 and IgG2b positive B cell population were then examined by flow cytometry (FIG. 5). A significant increase of subpopulation of IgG1 (FIG. 5a,b,c,g) and IgG2b (FIG. 5d,e,f,h) positive B cells was found in spleen cells co-cultured with platelets treated with Tat compared to those treated with Ad18-66.

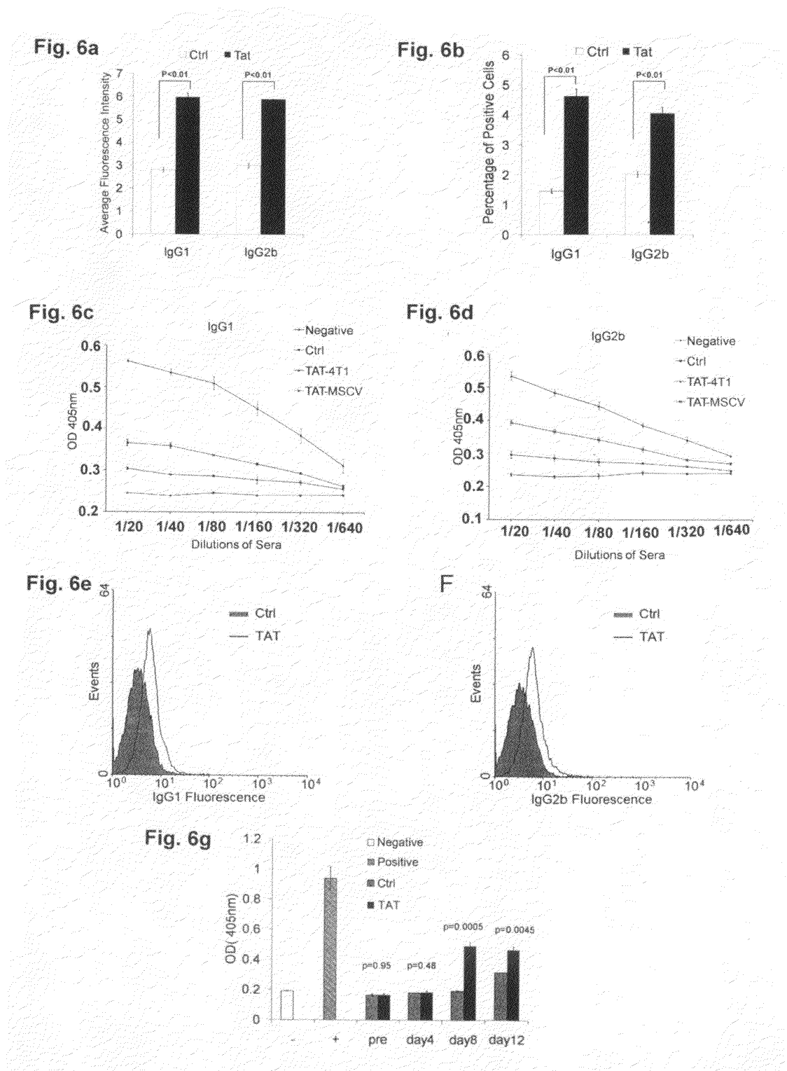

The present inventor next examined whether Tat-induced platelet activation and up-regulation of CD154 can affect immunoglobulin expression in vivo. Approximately a two-fold increase in IgG1 and IgG2b positive cells were found in 4T1-Tat cells injected mice (FIG. 6a, b). Serum levels of both IgG1 and IgG2b were increased approximately two-fold in both MSCV-Tat virus and 4T1-Tat cells injected mice (FIG. 6c, d). Thus, the data presented herein shows that Tat-induced platelet CD154 enhances B cell activity in vitro and in vivo.

Enhanced B Cell Antibody Response was Found in Tat-Treated Mouse Immunized with Adenovirus.

To assess the role of Tat-induced platelet activation in the B cell immune response in vivo, BALB/c mice were immunized with adenovirus and intravenously injected with Tat. Serum was collected at various time points. Positive IgG1 and IgG2b cells were monitored by flow cytometry and the titer of anti-adenovirus antibody was tested by ELISA. An increase of positive IgG1 and IgG2 cell population was found in mice injected with Tat (FIG. 6e, f). An early antibody response to adenovirus was seen in the group of mice injected with Tat at day 8 compared with the control mice at day 12 (FIG. 6g). Thus, results presented herein reveal that Tat-induced platelet activation and release of CD154 are involved in the early antibody response and potentially contribute the HIV-1 autoimmune disease, such as autoimmune thrombocytopenia.

### Electron Microscopy (EM) of Microparticles Released by TAT-Activated Platelets

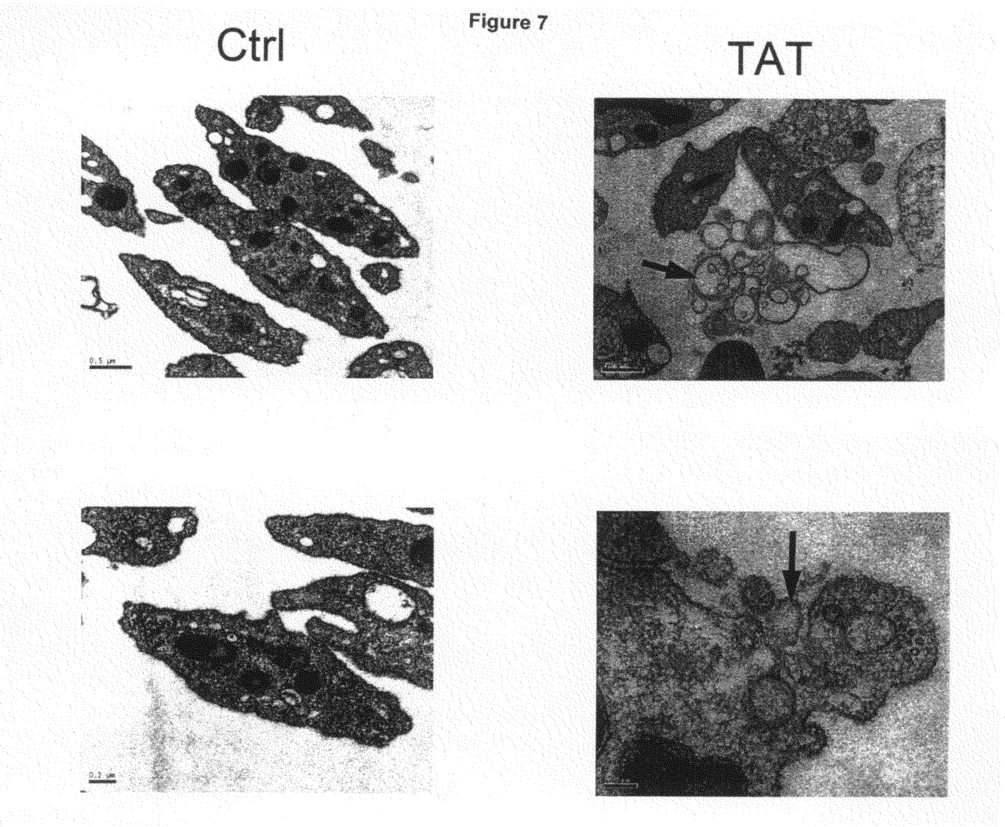

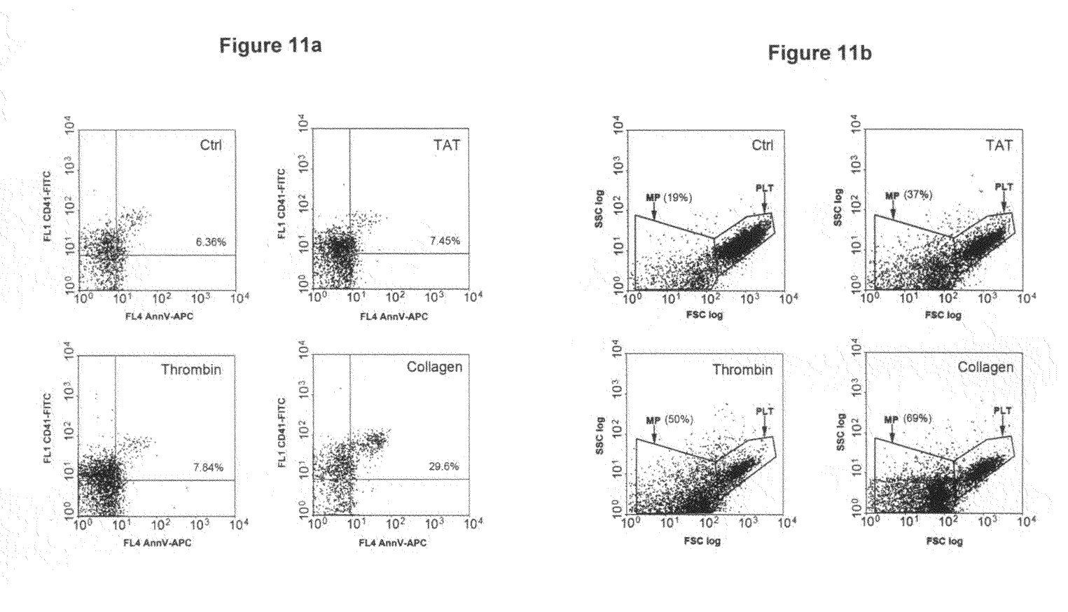

The release of platelet-derived microparticles (PMPs) is a sign of platelet activation. Chronic platelet activation leads to high levels of PMPs in peripheral blood and is associated with cardiovascular diseases (Tan et al. Ann Med. 2005; 37: 61-6). It has been shown recently that CD154 in PMPs is sufficient to facilitate the augmentation of adenovirus-specific antibodies (Sprague et al. Blood. 2008; 111: 5028-36). To test whether Tat is able to induce the release of PMPs in platelets, platelets were treated with Tat or Ad18-66 before fixation for electron microscopy. A significant number of PMP buds were seen at the plasma membrane of Tat-activated platelets (FIGS. 7 and 11). Thus, Tat is able to induce platelet activation and to release PMPs.

### Discussion

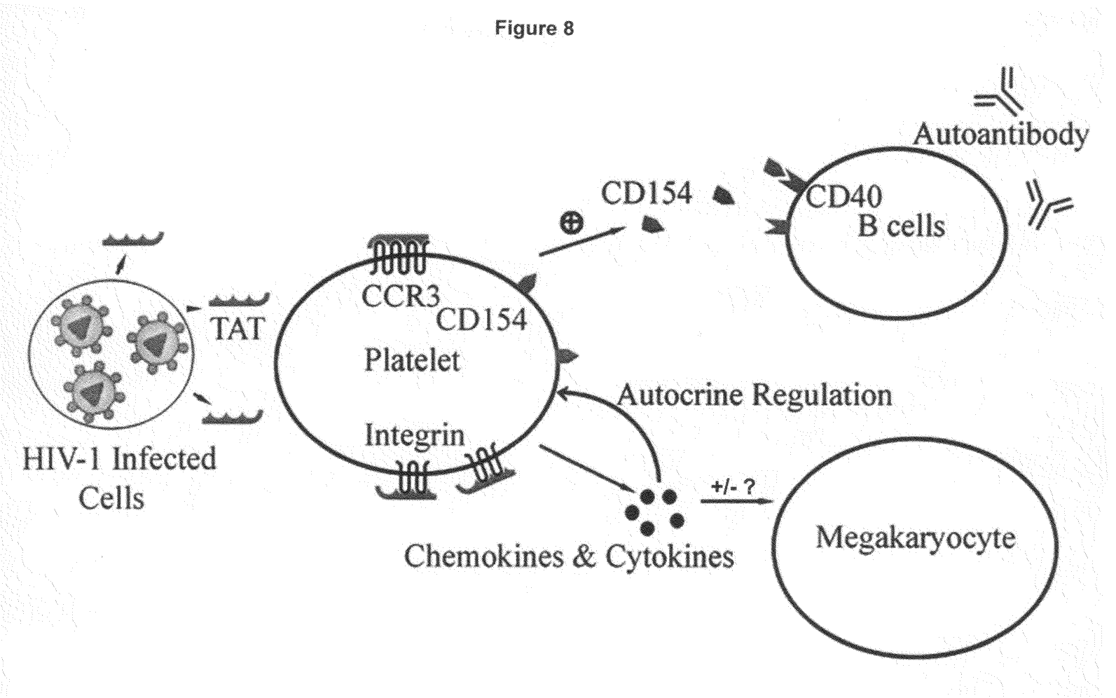

Two mechanisms may be involved in the HIV-1 induced platelet activation. One is via deregulated chemokines and cytokines. An alternative mechanism involves HIV derived protein(s) directly interacting with platelets (FIG. 8). HIV infected cells release cellular or HIV viral proteins (such as Tat or Nef) that effect bystander cells (Pugliese et al. Cell Biochem Funct. 2005; 23: 223-7; Fackler et al. Immunity. 2002; 16: 493-7). The extra-cellular functions of Tat and Nef have been studied intensively in several cell types. Tat is not only able to mimic the CCR3 ligand but also the integrin ligand with its RGD motif. Tat binds to integrin αVβ3 with an affinity similar to fibrinogen and vitronectin with dissociation constants (Kd) of 32 nM (Tat), 27 nM (fibrinogen) and 64 nM (vitronectin), respectively (Urbinati et al. Arterioscler Thromb Vasc Biol. 2005; 25: 2315-20). Due to the unique character of Tat protein, the interaction between Tat and platelet and its potential role in HIV-1 ITP was examined. Results presented herein demonstrate that Tat induced platelet activation requires both chemokine receptor CCR3 and integrin β3 (FIGS. 2 and 3). In addition, while calcium flux is required for Tat-induced platelet activation (FIG. 3j,k,l), PGE1 does not inhibit Tat-induced platelet activation, suggesting a limited role for cAMP in this process (FIG. 3g,h,i). Thus, the present inventor has described a new role of Tat on platelet activation. It is likely that Tat released by HIV infected cells (such as CD4⁺ T cells in the spleen) is able to first activate the platelets through CCR3 and then further activate platelets through integrin β3. Tat-activated platelets release platelet-derived micro-particle (PMPs) containing CD154, and PMPs may increase the activity of B cells and contribute to autoimmune response.

CD154 can play opposing roles in HIV infection (Kornbluth. J Leukoc Biol. 2000; 68: 373-82). On one hand, it promotes the immune response to HIV. On the other hand, it can activate CD4⁺ T cells through the activation of dendritic cells and macrophages to help HIV replication. In fact, it has been shown that macrophages stimulated by CD154 release sCD23 and sICAM to render T cells permissive to HIV-1 infection (Kornbluth. J Leukoc Biol. 2000; 68: 373-82; Swingler et al. Nature. 2003; 424: 213-9; Martin et al. J Virol. 2007; 81: 5872-81). Thus, it is possible platelet released CD154 is required for effective viral replication. Since anti-Tat antibodies frequently develop in HIV-infected patients, it needs to be determined whether anti-Tat antibody has any effect on Tat-induced platelet activation in HIV-infected patients (Re et al. Clin Diagn Lab Immunol. 1996; 3: 230-2; Re et al. J Clin Virol. 2001; 21: 81-9).

Antiretroviral therapy (ART) improves the thrombocytopenia while frequently inducing platelet activation in HIV-infected patients. Although the mechanism of ART-induced platelet activation is different from Tat-induced platelet activation, ART is frequently accompanied by increased cardiovascular disease and myocardial infarction in HIV-infected patients (Phillips et al. AIDS. 2008; 22: 2409-18. Thus, ART-induced platelet activation represents another aspect of HIV associated platelet activation that deserves further study (Satchell et al. AIDS. 24: 649-57).

Platelet agonists can be broadly grouped into two groups depending on their effectiveness in inducing platelet activation: strong agonists capable of inducing stable aggregation and weak agonists causing brief, unstable aggregation requiring a second agonist to induce complete aggregation. Thrombin and ADP are classic strong agonists, whereas epinephrine and serotonin are examples of weak agonists. Chemokines induce weak platelet activation. The present inventor found that Tat- as well as CCL5-induced platelet activation require both CCR3 and integrin β3, suggesting that a synergy of two receptor signals may be required for these two weak platelet receptors. Since a variety of platelet agonists only induces weak platelet activation, it is not clear what the biological significance of such activation is. Results presented herein demonstrate that one consequence is the presentation of CD154, which can have important immunomodulator effects. Whether the property of moderate platelet activation to serve as a source of additional reactive molecules (chemokines, cytokines etc.) represents another important aspect of platelet function is yet to be determined.

In conclusion, results presented herein demonstrate that: 1. Tat is able to induce the release of platelet CD154 and microparticles. 2. Tat-induced platelet activation requires chemokine receptor CCR3 and 133 integrin. 3. Tat-induced platelet activation is cAMP independent and calcium flux dependent. 4. Tat expression cell line 4T1 and TAT-MSCV retrovirus induce platelet activation in vivo. 5. Platelet derived CD154 stimulates B cell activity by enhancing Ig production. Therefore, data presented herein suggest that Tat-induced platelet CD154 may play a role in and contribute to the development of HIV-1 ITP. Additional studies will help us understand better the implications of HIV-1 Tat protein and platelet activation in the development of HIV-1 ITP.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for treating a patient with an infectious disease, the method comprising administering to the patient a therapeutically effective amount of a Trans Activating Factor (Tat) polypeptide or a composition comprising a Tat polypeptide, wherein administration of the Tat polypeptide or composition enhances immune responses to an infectious/etiological agent that causes the infectious disease.

2. The method of claim 1, wherein the infectious disease is caused by a virus that does not express the Tat polypeptide.

3. The method of claim 2, wherein the virus that does not express Tat polypeptide is a hepatitis virus, a herpesviruses, or an influenza virus.

4. The method of claim 1, wherein the infectious disease is caused by a microbe.

5. The method of claim 4, wherein the microbe is a bacteria.

6. The method of claim 1, wherein the Tat polypeptide comprises the amino acid sequence of SEQ ID NO: 2 or a derivative or truncation thereof.

7. The method of claim 1, further comprising administering an anti-viral agent to the patient.

8. The method of claim 7, wherein the anti-viral agent is acyclovir, valacyclovir, famciclovir, ganciclovir, amantadine, or rimantadine.

9. The method of claim 1, further comprising administering an anti-bacterial agent to the patient.

10. The method of claim 9, wherein the anti-bacterial agent is an antibiotic.

11. A composition comprising a Tat polypeptide and an anti-bacterial agent, wherein the Tat polypeptide comprises the amino acid sequence of SEQ ID NO: 2 or a derivative or truncation thereof.

12. An isolated amino acid sequence comprising a Tat polypeptide or peptide comprising the amino acid sequence of any one of the sequences shown in FIG. 12 or FIG. 13, wherein the Tat polypeptide or peptide stimulates platelet activation.

13. An expression vector encoding an amino acid sequence of claim 12, wherein expression of the amino acid sequence is controlled by regulatory sequences in the expression vector.

14. A composition comprising the isolated amino acid sequence of claim 12 and a pharmaceutically acceptable buffer.

15. An isolated nucleic acid sequence which encodes the Tat polypeptide or peptide of claim 12.

16. An expression vector comprising a nucleic acid sequence of claim 15, wherein said nucleic acid sequence is operably linked to a regulatory sequence.

17. A composition comprising the expression vector of claim 16 and a pharmaceutically acceptable buffer.

18. A method for treating a patient with an infectious disease, the method comprising administering to the patient a therapeutically effective amount of a composition of claim 14, wherein administration of the composition enhances immune responses to an infectious/etiological agent that causes the infectious disease.

19. The method of claim 20, wherein the infectious disease is a viral, bacterial, or fungal infection.

20. The method of claim 19, wherein the bacterial infection comprises at least one antibiotic resistant bacterial strain.
